(19) United States
(12) Patent Application Publication
(10) Pub. No.: US 2006/0110451 A1
(43) Pub. Date: May 25, 2006

(54) Formulations of [1,4]diazepino[6,7,1-IJ]quinoline derivatives

(75) Inventors: Yanning Lin, Lake Hiawatha, NJ (US); Wendy A. Dulin, Tuxedo, NY (US); Mannching Sherry Ku, Thiells, NY (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/267,605

(22) Filed: Nov. 4, 2005

Related U.S. Application Data
(60) Provisional application No. 60/625,280, filed on Nov. 5, 2004.

(51) Int. Cl. A61K 31/551; A61K 9/20

(52) U.S. Cl. 424/464; 514/220

(57) ABSTRACT

The present invention provides solid dosage formulations of [1,4]diazepino[6,7,1-ij]quinoline derivatives and processes for their manufacture. In some particular embodiments, the present invention provides novel formulations of the antipsychotic and antiobesity agent (9aR,12aS)-4,5,6,7,9,9a,10,11,12,12a-decahydrocyclopenta[c][1,4]diazepino[6,7,1-ij]quinoline hydrochloride (Compound A.HCl).

### CROSS REFERENCE TO RELATED APPLICATIONS

This application claims priority benefit of U.S. Provisional Application Ser. No. 60/625,280, filed Nov. 5, 2004, the entire disclosure of which is incorporated by reference herein in its entirety.

### FIELD OF THE INVENTION

The present invention relates to solid dosage formulations of [1,4]diazepino[6,7,1-ij]quinoline derivatives and processes for their manufacture. In some particular embodiments, the present invention provides novel formulations of the antipsychotic and antiobesity agent (9aR,12aS)-4,5,6,7,9,9a,10,11,12,12a-decahydrocyclopenta[c][1,4]diazepino[6,7,1-ij]quinoline hydrochloride (Compound A.HCl).

### BACKGROUND OF THE INVENTION

Schizophrenia affects approximately 5 million people. At present, the most widespread treatments for schizophrenia are the ‘atypical’ antipsychotics, which combine dopamine (D2) receptor antagonism with serotonin (5-HT_{2A}) receptor antagonism. Despite the reported advances in efficacy and side-effect liability of atypical antipsychotics over typical antipsychotics, these compounds do not adequately treat all of the symptoms of schizophrenia and are accompanied by problematic side effects including weight gain (Allison, D. B., et. al., Am. J. Psychiatry, 156:1686-1696, 1999; Masand, P. S., Exp. Opin. Pharmacother. 1: 377-389, 2000; Whitaker, R., Spectrum Life Sciences. Decision Resources. 2:1-9, 2000). Novel antipsychotics which are effective in treating the mood disorders or the cognitive impairments in schizophrenia without producing weight gain would represent a significant advance in the treatment of schizophrenia.

5-HT_{2C} agonists and partial agonists represent a novel therapeutic approach toward the treatment of schizophrenia. Several lines of evidence support a role for 5-HT_{2C} receptor agonism as a treatment for schizophrenia. Studies with 5-HT_{2C} antagonists suggest that these compounds increase synaptic levels of dopamine and may be effective in animal models of Parkinson's disease (Di Matteo, V., et. al., Neuropharmacology 37: 265-272, 1998; Fox, S. H., et. al., Experimental Neurology 151: 35-49, 1998). Since the positive symptoms of schizophrenia are associated with increased levels of dopamine, compounds with actions opposite those of 5-HT_{2C} antagonists such as 5-HT_{2C} agonists and partial agonists should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology 37: 953-955, 1998; Di Matteo, V., et. al., Neuropharmacology 38: 1195-1205, 1999; Di Giovanni, G., et. al., Synapse 35: 53-61, 2000), brain regions that are thought to mediate critical antipsychotic effects of drugs like clozapine. In contrast, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in substantia nigra. The differential effects of 5-HT_{2C} agonists in the mesolimbic pathway relative to the nigrostriatal pathway suggests that 5-HT_{2C} agonists will have limbic selectivity and will be less likely to produce extrapyramidal side effects associated with typical antipsychotics.

Atypical antipsychotics bind with high affinity to 5-HT_{2C} receptors and function as 5-HT_{2C} receptor antagonists or inverse agonists. Weight gain is a problematic side effect associated with atypical antipsychotics such as clozapine and olanzapine and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology 124: 57-73, 1996; Cowen, P. J., et. al., Human Psychopharmacology 10: 385-391, 1995; Rosenzweig-Lipson, S., et. al., ASPET abstract, 2000). As a result, 5-HT_{2C} agonists and partial agonists will be less likely to produce the body weight increases associated with current atypical antipsychotics. Indeed, 5-HT_{2C} agonists and partial agonists are of great interest for the treatment of obesity, a medical disorder characterized by an excess of body fat or adipose tissue and associated with such comorbidities as Type II diabetes, cardiovascular disease, hypertension, hyperlipidemia, stroke, osteoarthritis, sleep apnea, gall bladder disease, gout, some cancers, some infertility, and early mortality.

The compound (9aR,12aS)-4,5,6,7,9,9a,10,11,12,12a-decahydrocyclopenta[c][1,4]diazepino[6,7,1-ij]quinoline hydrochloride (Compound A.HCl):

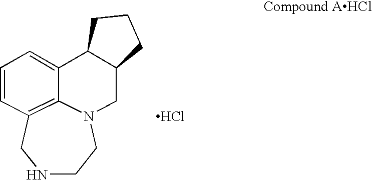

is a potent 5-HT_{2C} agonist. See U.S. patent application Ser. No. 10/422,524, filed Apr. 24, 2003, and International Application WO 03/091250, each of which is incorporated by reference herein in its entirety. Compound A can also be effective in treating the mood disorders or the cognitive impairments associated with schizophrenia.

Given the importance of compounds such as Compound A as pharmaceutical agents, it can be seen that effective formulations for their delivery is of great import. This invention is directed to these, as well as other, important ends.

### SUMMARY OF THE INVENTION

In one aspect, the present invention provides pharmaceutical compositions comprising a compound of Formula I as described herein, preferably (9aR,12aS)-4,5,6,7,9,9a,10,11,12,12a-decahydrocyclopenta[c][1,4]diazepino[6,7,1-ij]quinoline hydrochloride (Compound A.HCl), in an enteric coated form. In further embodiments, the present invention provides such compositions that further include a sustained release component, that can include one or more release-controlling excipients.

In some embodiments, the present invention provides pharmaceutical compositions comprising:

a) a pharmaceutically effective amount of an active pharmacological agent comprising from about 10% to about 80% by weight of the pharmaceutical composition;

b) a filler component comprising from about 10% to about 80% by weight of the pharmaceutical composition; c) an optional seal coat component comprising from about 0.01% to about 5% by weight of the pharmaceutical composition;

d) an enteric coat component comprising from about 0.01% to about 20% by weight of the pharmaceutical composition;

e) an optional glidant component comprising from about 0.01% to about 20% by weight of the pharmaceutical composition;

f) an optional plasticizer component comprising from about 0.01% to about 3% by weight of the pharmaceutical composition;

g) an optional neutralizer component comprising from about 0.01% to about 1.5% by weight of the pharmaceutical composition;

h) an optional surfactant component comprising from about 0.001% to about 1.0% by weight of the pharmaceutical composition; and

i) an optional lubricant component comprising from about 0.01% to about 5.0% by weight of the pharmaceutical composition;

wherein the active pharmacological agent has the Formula I: 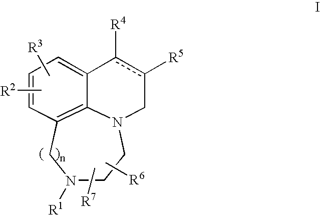
wherein:
R¹ is hydrogen, alkyl of 1 to 6 carbon atoms, alkanoyl of 2 to 6 carbon atoms, or carboarylalkoxy of 7 to 11 carbon atoms;
R² and R³ are each, independently, hydrogen, hydroxy, alkyl of 1-6 carbon atoms, alkoxy of 1-6 carbon atoms, halogen, carboxamido, carboalkoxy of two to six carbon atoms, perfluoroalkyl of 1-6 carbon atoms, cyano, alkanesulfonamido of 1-6 carbon atoms, alkanesulfonyl of 1-6 carbon atoms, alkanamido of 1-6 carbon atoms, amino, alkylamino of 1-6 carbon atoms, dialkylamino of 1-6 carbon atoms per alkyl moiety, perfluoroalkoxy of 1-6 carbon atoms, alkanoyloxy of 2 to 6 carbon atoms, alkanoyl of 2 to 6 carbon atoms, aroyl of 6 to 8 carbon atoms, aryl of 5 to 7 carbon atoms, a C₆ to C₁₃ alkylaryl group having 5 to 7 carbon atoms in the aryl moiety, a 5 to 7 membered heteroaryl group, or a 6 to 13 membered alkylheteroaryl group having 5 to 7 members in the heteroaryl moiety, wherein any R² or R³ substituent having an aryl or heteroaryl moiety may optionally be substituted on the aryl or heteroaryl moiety with 1 to 3 substituents independently selected from a halogen atom, a C₁-C₆ alkyl group, or a C₁-C₆ alkoxy group;
R⁴ and R⁵ are, independently, hydrogen or alkyl of 1 to 6 carbon atoms, or R⁴ and R⁵, taken together with the carbons to which they are attached, form a cyclic moiety selected from a cycloalkane of 4 to 8 carbon atoms, cycloalkene of 4 to 8 carbon atoms, bridged bicyclic alkane of 5 to 10 carbon atoms, bridged bicyclic alkene of 5 to 10 carbon atoms, pyran or thiopyran in which the sulfur atom is optionally oxidized to the sulfoxide or sulfone, wherein the cyclic moiety formed by R⁴ and R⁵ may optionally be substituted with 1 to 3 substituents independently selected from a halogen atom, a C₁-C₆ alkyl group, or a C₁-C₆ alkoxy group;
R⁶ and R⁷ are each, independently, hydrogen or alkyl of 1 to 6 carbon atoms;
   n is 1 or 2; and
   a dotted line represents an optional double bond;
   or a pharmaceutically acceptable salt thereof.

In some embodiments, R² is hydrogen, halogen, cyano, perfluoroalkyl of 1 to 3 carbon atoms, alkyl of 1 to 6 carbon atoms, alkoxy of 1 to 6 carbon atoms, alkanoyl of 2 to 6 carbon atoms, alkanesulfonyl of 1 to 6 carbon atoms, or aryl of 5 to 7 carbon atoms. In some further embodiments, R² is hydrogen, halogen, cyano, alkoxy of 1 to 3 carbon atoms, phenyl or trifluoromethyl. In still further embodiments, R³ is hydrogen, halogen, cyano, perfluoroalkyl of 1 to 3 carbon atoms, alkyl of 1 to 6 carbon atoms, alkoxy of 1 to 6 carbon atoms, alkanoyl of 2 to 6 carbon atoms, alkanesulfonyl of 1 to 6 carbon atoms, or aryl of 5 to 7 carbon atoms.

In some further embodiments, R³ is hydrogen, halogen, cyano, alkoxy of 1 to 3 carbon atoms, phenyl or trifluoromethyl. In further embodiments, R⁴ and R⁵ are preferably taken together, along with the carbon atoms to which they are attached, to form a cycloalkane or cycloalkene moiety of 5 to 8 carbon atoms, where one or more of the carbon atoms are optionally substituted by alkyl of 1 to 4 carbon atoms. In still further embodiments, R⁴ and R⁵ are preferably taken together, along with the carbon atoms to which they are attached, to form a cycloalkane moiety of 5 to 7 carbon atoms. In some embodiments, R⁶ and R⁷ are each hydrogen. In further embodiments, n is 1. In some further embodiments, R² and R³ are independently selected from hydrogen, halo, trifluoromethyl, phenyl or alkoxy of 1 to 3 carbon atoms, R¹, R⁶ and R⁷ are each hydrogen, n is 1, and R⁴ and R⁵, taken together with the carbon atoms to which they are attached, form cyclopentane, cyclohexane or cycloheptane. In some particularly preferred embodiments, the active pharmacological agent is Compound A, or a pharmaceutically acceptable salt thereof, preferably Compound A.HCl.

In some embodiments:

the active pharmacological agent comprises from about 15% to about 25% by weight, or from about 20% to about 22% by weight, or from about 20% to about 40% by weight, or from about 30% to about 40% by weight, of the pharmaceutical composition;

the filler component comprises from about 50% to about 70% by weight, or from about 60% to about 66% by weight, or from about 40% to about 70% by weight, or from about 40% to about 50% by weight, of the pharmaceutical composition;

the optional seal coat component, when present, comprises from about 0.5% to about 3% by weight, or from about 1% to about 3% by weight, or from about 1% to about 2% by weight, or from about 2% to about 3% by weight, of the pharmaceutical composition;

the enteric coat component comprises from about 5% to about 15% by weight, or from about 8% to about 12% by weight, or from about 8% to about 16% by weight, or from about 12% to about 16% by weight, or from about 9% to about 11% by weight, or from about 9.6% to about 10.6% by weight, or from about 10.0% to about 10.2% by weight, of the pharmaceutical composition;

the optional glidant component, when present, comprises from about 0.01% to about 1% by weight, or from about 0.1% to about 0.3% by weight, or from about 0.1% to about 3% by weight, of the pharmaceutical composition;

the optional plasticizer component, when present, comprises from about 0.1% to about 1.5% by weight, or from about 0.5% to about 1.0% by weight, or from about 0.5% to about 2.0% by weight, of the pharmaceutical composition;

the optional neutralizer component, when present, comprises from about 0.01% to about 0.8% by weight, or from about 0.05% to about 0.3% by weight, or from about 0.03% to about 0.3% by weight, of the pharmaceutical composition;

the optional surfactant component, when present, comprises from about 0.005% to about 0.05% by weight, or from about 0.005% to about 0.025% by weight, or from about 0.001% to about 0.3% by weight, of the pharmaceutical composition; and

the optional lubricant component, when present, comprises from about 1% to about 4% by weight, or from about 2.5% to about 3.5% by weight, or from about 2% to about 3.5% by weight, of the pharmaceutical composition.

In some further embodiments:

a) the active pharmacological agent comprises from about 30% to about 80% by weight of the pharmaceutical composition, or from about 30% to about 45% by weight of the pharmaceutical composition, or from about 50% to about 70% by weight of the pharmaceutical composition, or from about 60% to about 70% by weight of the pharmaceutical composition;

b) the filler component comprises from about 40% to about 60% by weight of the pharmaceutical composition, or from about 10% to about 30% by weight of the pharmaceutical composition;

c) the optional seal coat component, when present, comprises from about 0.5% to about 3% by weight of the pharmaceutical composition; d) the enteric coat component comprises from about 5% to about 15% by weight of the pharmaceutical composition;

e) the optional glidant component, when present, comprises from about 0.1% to about 2% by weight of the pharmaceutical composition;

f) the optional plasticizer component, when present, comprises from about 0.1% to about 1.5% by weight of the pharmaceutical composition;

g) the optional neutralizer component, when present, comprises from about 0.01% to about 0.8% by weight of the pharmaceutical composition;

h) the optional surfactant component, when present, comprises from about 0.005% to about 0.05% by weight of the pharmaceutical composition; and

i) the optional lubricant component, when present, comprises from about 1% to about 4% by weight of the pharmaceutical composition.

In some embodiments, the glidant component, the plasticizer component, the neutralizer component, the surfactant component, and the lubricant component are each present in the formulation.

In some embodiments, the filler component includes one or more of microcrystalline cellulose, lactose, starch, carboxymethyl cellulose, cellulose gum, polyethylene glycol, substituted celluloses, ethyl cellulose, carboxyethyl cellulose, hydroxyethyl celluloses, calcium phosphates, anhydrous dicalcium phosphate, metal aluminosilicates, magnesium aluminometasilicate (e.g., Neusilin®), sugar or carbohydrate containing compounds, mannitol, sucrose, maltodextrin, sorbitol, starch, xylitol, metal phosphates, metal carbonates, and magnesium carbonate. In some preferred embodiments, the filler includes microcrystalline cellulose.

In some embodiments, the optional seal coat component, when present, includes one or more of Opadry® II Clear, other Opadry® coat materials, Kollicoat®, maltodextrin, Pure-Cote®, Pharmacoat®, or other cellulose- or starch-based coat.

In some embodiments, the enteric coat component includes one or more of methacrylate copolymer, where the copolymer has a functional group at a pH that is anionic such as Eudragit® L30D-55 dry polymer or cationic such as the Eudragit® aminoalkyl methacrylate copolymers, or where the copolymer has no functional group, i.e., is pH-independent and thus, is neutral such as Eudragit® NE 30 D/40 D; a methacrylic acid copolymer such as Acryl-EZE®; a HPMC containing enteric coating system such as Spectrablend™; CAP; HPMCP; an acrylic polymer such as Eastacryl®, or other acrylate-, methacrylate- or cellulose acetate phthalate-based coat. In some preferred embodiments, the enteric coat component includes Eudragit® L30D-55 dry polymer. In such embodiments, the copolymer, polymer or coating system can be in a variety of forms such as granules, a solid substance, a dispersion, or an organic solution.

In some embodiments, the optional glidant component, when present, includes one or more of mono- and di-glycerides, talc, silicon dioxide, stearic acid, starch powdered cellulose, lactose, stearates, calcium phosphates, magnesium carbonate, magnesium oxide, silicates, and silicon dioxide aerogels. In some preferred embodiments, the glidant includes mono- and di-glycerides, for example Imwitor® 900K.

In some embodiments, the optional plasticizer component, when present, includes one or more of triethyl citrate, dibutyl sebecate, polyethylene glycol, propylene glycol, triacetin, sorbitol, tributyl citrate, acetyltributyl citrate, acetyltriethyl citrate, dibutyl phthalate, triethyl citrate and triethanolamine. In some preferred embodiments, the plasticizer component includes triethyl citrate.

In some embodiments, the optional neutralizer component, when present, includes one or more of NaOH, KOH, and NH₄OH. In some preferred embodiments, the plasticizer component includes NaOH.

In some embodiments, the optional surfactant component, when present, includes one or more of polysorbate 80, sodium lauryl sulfate, sucrose palmitate, poloxamer, docusate sodium, and polyoxyethylene sorbitan fatty acid esters, polyoxyethylene stearates, sucrose fatty acid esters and sorbitan fatty acid esters. In some preferred embodiments, the surfactant component includes polysorbate 80.

In some embodiments, the optional lubricant component, when present, includes one or more of talc, metallic stearates, silicon dioxide, sodium stearyl fumarate, fatty acid esters, fatty acids, fatty alcohols, glyceryl behenate, mineral oil, paraffins, hydrogenated vegetable oils, leucine, polyethylene glycols, metallic lauryl sulfates, silica such as Aerosil® 200, and sodium chloride. In some preferred embodiments, the lubricant component includes talc.

In some embodiments, the active pharmacological agent comprises Compound A, or a pharmaceutically acceptable salt thereof, preferably Compound A HCl, the filler component includes microcrystalline cellulose; the seal coat component includes Opadry® II clear; the enteric coat component includes Eudragit® L30D-55 dry polymer; the glidant component includes mono- and di-glycerides; the plasticizer component includes triethyl citrate; the neutralizer component includes NaOH; the surfactant component includes polysorbate 80; and the lubricant component includes talc.

In some embodiments of each of the foregoing compositions, the composition comprises a plurality of enteric-coated pellets. In some such embodiments, the enteric-coated pellets are present in a capsule.

The present invention further provides processes for preparing the disclosed pharmaceutical compositions, comprising:

a) preparing uncoated pellets comprising said filler and said active pharmacological agent;

b) optionally applying a subcoat to the uncoated pellets; and

c) applying an enteric coating to the pellets.

In some embodiments, the processes further comprise the step of:

d) filling a capsule with said pellets to achieve a predetermined dose of Compound A, or a pharmaceutically acceptable salt thereof, preferably Compound A.HCl.

In some embodiments, step (a) includes:

i) mixing the filler and the active pharmacological agent to form a mixture;

ii) wet granulating the mixture to form a granulate; and

iii) extruding and spheronizing the granulate.

In some embodiments, step (c) includes:

i) preparing a suspension comprising said enteric coat, said plasticizer, said neutralizer, and said surfactant; and

ii) spraying said suspension onto said pellets.

The invention also provides products of the processes described herein.

In some embodiments, the invention provides pharmaceutical compositions comprising a plurality of enteric coated pellets, said pellets comprising:

a) an active pharmacological agent comprising from about 20% to about 22% by weight of the pharmaceutical composition;

b) a filler component comprising from about 60% to about 66% by weight of the pharmaceutical composition;

c) an optional seal coat component comprising, when present, from about 1% to about 2% by weight of the pharmaceutical composition;

d) an enteric coat component comprising from about 8% to about 12% by weight of the pharmaceutical composition;

e) an optional glidant component comprising, when present, from about 0.1% to about 0.3% by weight of the pharmaceutical composition;

f) an optional plasticizer component comprising, when present, from about 0.5% to about 1.0% by weight of the pharmaceutical composition;

g) an optional neutralizer component comprising, when present, from about 0.05% to about 0.3% by weight of the pharmaceutical composition;

h) an optional surfactant component comprising, when present, from about 0.005% to about 0.025% by weight of the pharmaceutical composition; and

i) an optional lubricant component comprising, when present, from about 2.5% to about 3.5% by weight of the pharmaceutical composition;

wherein:

the active pharmacological agent comprises Compound A, or a pharmaceutically acceptable salt thereof, preferably Compound A.HCl;

the filler component comprises microcrystalline cellulose;

the seal coat component, when present, comprises, Opadry® II Clear;

the enteric coat component comprises a methacrylic copolymer such as Eudragit® L30D-55;

the glidant component, when present, comprises mono- and di-glycerides;

the plasticizer component, when present, comprises triethyl citrate;

the neutralizer component, when present, comprises NaOH;

the surfactant component, when present, comprises polysorbate 80; and

the lubricant component, when present, comprises talc.

In some embodiments, each of said components (c), (e), (f), (g), (h) and (i) are present.

In some embodiments of each of the foregoing pharmaceutical compositions and processes, the composition contains from about 0.5 mg to about 5.0 mg of active pharmacological agent, or from about 1.0 mg to about 3.0 mg of active pharmacological agent, or from about 1.5 mg to about 2.5 mg of active pharmacological agent, or from about 20 mg to about 30 mg of active pharmacological agent, or from about 22.5 mg to about 27.5 mg of active pharmacological agent, or from about 24.0 mg to about 26.0 mg of active pharmacological agent, or from about 50 mg to about 100 mg of active pharmacological agent, or from about 65 mg to about 85 mg of active pharmacological agent, or from about 70 mg to about 80 mg of active pharmacological agent, or from about 75 mg to about 125 mg of active pharmacological agent, or from about 90 mg to about 110 mg of active pharmacological agent, or from about 72 mg to about 76 mg of active pharmacological agent, or from about 20 mg to about 110 mg of active pharmacological agent.

In some further embodiments, the composition contains from about 50.0 mg to about 750.0 mg of active pharmacological agent, or from about 50.0 mg to about 200.0 mg of active pharmacological agent, or from about 100.0 mg to about 175.0 mg of active pharmacological agent, or from about 125.0 mg to about 175.0 mg of active pharmacological agent.

In some further embodiments, the composition contains from about 100.0 mg to about 750.0 mg of active pharmacological agent, or from about 150.0 mg to about 750.0 mg of active pharmacological agent, or from about 200.0 mg to about 750.0 mg of active pharmacological agent, or from about 300.0 mg to about 750.0 mg of active pharmacological agent. In some embodiments, the composition contains about 2 mg, about 4 mg, about 10 mg, about 25 mg, about 50 mg, about 75 mg, about 100 mg, about 150 mg, about 200 mg, about 300 mg, about 400 mg, about 450 mg, about 500 mg, about 600 mg, about 700 mg, or about 750 mg of active pharmacological agent.

In some embodiments of each of the pharmaceutical compositions described herein, the compositions can further include a sustained release component. In some embodiments, the sustained release component is a coating that is disposed in between the optional seal coat component and the enteric coat component. In some embodiments, the sustained release coating comprises a release-controlling excipient, for example one or more of gelatin, shellac, hydroxypropylmethyl cellulose (HPMC), methylcellulose (MC), ethylcellulose (EC), hydroxypropylmethyl cellulose phthalate (HPMCP), cellulose acetate phthalate (CAP), methacrylic acid/methyl methacrylate copolymers, polyvinyl acetate phthalate (PVAP), glyceryl behenate, paraffin or carnauba wax.

### BRIEF DESCRIPTION OF THE FIGURES

FIG. 1 shows Compound A mean plasma concentration versus time profiles for after single dose administration of 150 mg Compound A as a solution in comparison to an enteric dosage form according to the invention.

FIG. 2 shows Compound A.HCl plasma concentration (mean±SEM) vs. time profiles in healthy subjects receiving single and multiple (q12h) oral doses of 25, 50, 75, 100, 150 or 250 mg Compound A.HCl.

FIG. 3 shows Compound A.HCl trough plasma concentrations in healthy subjects receiving multiple (q12h) oral doses of 25, 50, 75, 100, 150 or 250 mg Compound A.HCl.

FIG. 4A shows the relationship between Compound A.HCl Cₘₐₓ and dose in healthy young subjects receiving single and multiple (q12h) oral doses of 25, 50, 75, 100, 150, or 250 mg Compound A.HCl.

FIG. 4B shows the relationship between Compound A.HCl AUC and dose in healthy young subjects receiving single and multiple (q12h) oral doses of 25, 50, 75, 100, 150, or 250 mg Compound A.HCl.

FIG. 5 shows Compound A.HCl plasma concentration (mean±SEM) vs. time profiles in schizophrenic patients receiving single and multiple (q12h) oral doses of 100, 150 or 250 mg Compound A.HCl.

FIG. 6 shows Compound A.HCl trough plasma concentrations in schizophrenic patients receiving multiple (q12h) oral doses of 100, 150 or 250 mg Compound A.HCl.

FIG. 7A shows the relationship between Compound A.HCl Cₘₐₓ and dose in schizophrenic patients receiving single and multiple (q12h) oral doses of 100, 150, or 250 mg Compound A.HCl.

FIG. 7B shows the relationship between Compound A.HCl AUC and dose in schizophrenic patients receiving single and multiple (q12h) oral doses of 100, 150, or 250 mg Compound A.HCl.

### DETAILED DESCRIPTION

During early toxicology testing, an emetic reaction to Compound A.HCl was noted in dogs when the drug was given as an oral suspension. Enteric-coated capsules were developed which delayed the release of the drug. It was found that this formulation improved the tolerability and allowed higher dosing in toxicology studies with dogs. For human clinical studies, enteric-coated pellets were developed which provided advantages over enteric-coated capsules for dose flexibility and more uniform release in the intestinal tract.

Thus, the present invention provides pharmaceutical compositions comprising [1,4]diazepino[6,7,1-ij]quinoline derivatives, and particularly the antipsychotic and antiobesity agent Compound A, or a pharmaceutically acceptable salt thereof, preferably Compound A.HCl. In some preferred embodiments, the compositions of the invention are delay-release formulations. In some such embodiments, the delay-release is accomplished with an enteric coating. In some embodiments, the composition includes enteric-coated pellets.

In one embodiment, the present invention provides pharmaceutical compositions comprising:

a) a pharmaceutically effective amount of an active pharmacological agent comprising from about 10% to about 80% by weight of the pharmaceutical composition;

b) a filler component comprising from about 10% to about 80% by weight of the pharmaceutical composition;

c) an optional seal coat component comprising from about 0.01% to about 5% by weight of the pharmaceutical composition;

d) an enteric coat component comprising from about 0.01% to about 20% by weight of the pharmaceutical composition;

e) an optional glidant component comprising from about 0.01% to about 20% by weight of the pharmaceutical composition;

f) an optional plasticizer component comprising from about 0.01% to about 3% by weight of the pharmaceutical composition;

g) an optional neutralizer component comprising from about 0.01% to about 1.5% by weight of the pharmaceutical composition;

h) an optional surfactant component comprising from about 0.001% to about 1.0% by weight of the pharmaceutical composition; and

i) an optional lubricant component comprising from about 0.01% to about 5.0% by weight of the pharmaceutical composition;

wherein the active pharmacological agent has the Formula I: 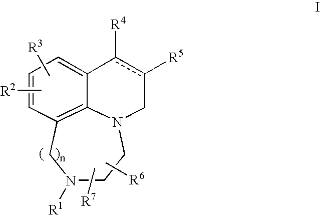
wherein:
R¹ is hydrogen, alkyl of 1 to 6 carbon atoms, alkanoyl of 2 to 6 carbon atoms, or carboarylalkoxy of 7 to 11 carbon atoms;
R² and R³ are each, independently, hydrogen, hydroxy, alkyl of 1-6 carbon atoms, alkoxy of 1-6 carbon atoms, halogen, carboxamido, carboalkoxy of two to six carbon atoms, perfluoroalkyl of 1-6 carbon atoms, cyano, alkanesulfonamido of 1-6 carbon atoms, alkanesulfonyl of 1-6 carbon atoms, alkanamido of 1-6 carbon atoms, amino, alkylamino of 1-6 carbon atoms, dialkylamino of 1-6 carbon atoms per alkyl moiety, perfluoroalkoxy of 1-6 carbon atoms, alkanoyloxy of 2 to 6 carbon atoms, alkanoyl of 2 to 6 carbon atoms, aroyl of 6 to 8 carbon atoms, aryl of 5 to 7 carbon atoms, a C₆ to C₁₃ alkylaryl group having 5 to 7 carbon atoms in the aryl moiety, a 5 to 7 membered heteroaryl group, or a 6 to 13 membered alkylheteroaryl group having 5 to 7 members in the heteroaryl moiety, wherein any R² or R³ substituent having an aryl or heteroaryl moiety may optionally be substituted on the aryl or heteroaryl moiety with 1 to 3 substituents independently selected from a halogen atom, a C₁-C₆ alkyl group, or a C₁-C₆ alkoxy group;
R⁴ and R⁵ are, independently, hydrogen or alkyl of 1 to 6 carbon atoms, or R⁴ and R⁵, taken together with the carbons to which they are attached, form a cyclic moiety selected from a cycloalkane of 4 to 8 carbon atoms, cycloalkene of 4 to 8 carbon atoms, bridged bicyclic alkane of 5 to 10 carbon atoms, bridged bicyclic alkene of 5 to 10 carbon atoms, pyran or thiopyran in which the sulfur atom is optionally oxidized to the sulfoxide or sulfone, wherein the cyclic moiety formed by R⁴ and R⁵ may optionally be substituted with 1 to 3 substituents independently selected from a halogen atom, a C₁-C₆ alkyl group, or a C₁-C₆ alkoxy group;
R⁶ and R⁷ are each, independently, hydrogen or alkyl of 1 to 6 carbon atoms;
   n is 1 or 2; and
   a dotted line represents an optional double bond;
   or a pharmaceutically acceptable salt thereof.

In some embodiments, R² is hydrogen, halogen, cyano, perfluoroalkyl of 1 to 3 carbon atoms, alkyl of 1 to 6 carbon atoms, alkoxy of 1 to 6 carbon atoms, alkanoyl of 2 to 6 carbon atoms, alkanesulfonyl of 1 to 6 carbon atoms, or aryl of 5 to 7 carbon atoms. In some further embodiments, R² is hydrogen, halogen, cyano, alkoxy of 1 to 3 carbon atoms, phenyl or trifluoromethyl. In still further embodiments, R³ is hydrogen, halogen, cyano, perfluoroalkyl of 1 to 3 carbon atoms, alkyl of 1 to 6 carbon atoms, alkoxy of 1 to 6 carbon atoms, alkanoyl of 2 to 6 carbon atoms, alkanesulfonyl of 1 to 6 carbon atoms, or aryl of 5 to 7 carbon atoms.

In some further embodiments, R³ is hydrogen, halogen, cyano, alkoxy of 1 to 3 carbon atoms, phenyl or trifluoromethyl. In further embodiments, R⁴ and R⁵ are preferably taken together, along with the carbon atoms to which they are attached, to form a cycloalkane or cycloalkene moiety of 5 to 8 carbon atoms, where one or more of the carbon atoms are optionally substituted by alkyl of 1 to 4 carbon atoms. In still further embodiments, R⁴ and R⁵ are preferably taken together, along with the carbon atoms to which they are attached, to form a cycloalkane moiety of 5 to 7 carbon atoms. In some embodiments, R⁶ and R⁷ are each hydrogen. In further embodiments, n is 1. In some further embodiments, R² and R³ are independently selected from hydrogen, halo, trifluoromethyl, phenyl or alkoxy of 1 to 3 carbon atoms, R¹, R⁶ and R⁷ are each hydrogen, n is 1, and R⁴ and R⁵, taken together with the carbon atoms to which they are attached, form cyclopentane, cyclohexane or cycloheptane.

In some particularly preferred embodiments, the active pharmacological agent is Compound A.HCl.

In some embodiments:

the active pharmacological agent comprises from about 15% to about 25% by weight, or from about 20% to about 22% by weight, or from about 20% to about 40% by weight, or from about 30% to about 40% by weight, of the pharmaceutical composition;

the filler component comprises from about 50% to about 70% by weight, or from about 60% to about 66% by weight, or from about 40% to about 70% by weight, or from about 40% to about 50% by weight, of the pharmaceutical composition;

the optional seal coat component, when present, comprises from about 0.5% to about 3% by weight, or from about 1% to about 3% by weight, or from about 1% to about 2% by weight, or from about 2% to about 3% by weight, of the pharmaceutical composition;

the enteric coat component comprises from about 5% to about 15% by weight, or from about 8% to about 12% by weight, or from about 12% to about 16% by weight, or from about 8% to about 16% by weight, or from about 9% to about 11% by weight, or from about 9.6% to about 10.6% by weight, or from about 10.0% to about 10.2% by weight, of the pharmaceutical composition;

the optional glidant component, when present, comprises from about 0.01% to about 1% by weight, or from about 0.1% to about 0.3% by weight, or from about 0.1% to about 3% by weight, of the pharmaceutical composition;

the optional plasticizer component, when present, comprises from about 0.1% to about 1.5% by weight, or from about 0.5% to about 1.0% by weight, or from about 0.5% to about 2.0% by weight, of the pharmaceutical composition;

the optional neutralizer component, when present, comprises from about 0.01% to about 0.8% by weight, or from about 0.05% to about 0.3% by weight, or from about 0.03% to about 0.3% by weight, of the pharmaceutical composition;

the optional surfactant component, when present, comprises from about 0.005% to about 0.05% by weight, or from about 0.005% to about 0.025% by weight, or from about 0.001% to about 0.3% by weight, of the pharmaceutical composition; and

the optional lubricant component, when present, comprises from about 1% to about 4% by weight, or from about 2.5% to about 3.5% by weight, or from about 2.0% to about 3.5% by weight, of the pharmaceutical composition.

In some further embodiments:

a) the active pharmacological agent comprises from about 30% to about 80% by weight of the pharmaceutical composition, or from about 30% to about 45% by weight of the pharmaceutical composition, or from about 50% to about 70% by weight of the pharmaceutical composition, or from about 60% to about 70% by weight of the pharmaceutical composition;

b) the filler component comprises from about 40% to about 60% by weight of the pharmaceutical composition, or from about 10% to about 30% by weight of the pharmaceutical composition;

c) the optional seal coat component, when present, comprises from about 0.5% to about 3% by weight of the pharmaceutical composition;

d) the enteric coat component comprises from about 5% to about 15% by weight of the pharmaceutical composition;

e) the optional glidant component, when present, comprises from about 0.1% to about 2% by weight of the pharmaceutical composition;

f) the optional plasticizer component, when present, comprises from about 0.1% to about 1.5% by weight of the pharmaceutical composition;

g) the optional neutralizer component, when present, comprises from about 0.01% to about 0.8% by weight of the pharmaceutical composition;

h) the optional surfactant component, when present, comprises from about 0.005% to about 0.05% by weight of the pharmaceutical composition; and

i) the optional lubricant component, when present, comprises from about 1% to about 4% by weight of the pharmaceutical composition.

In some embodiments, the glidant component, the plasticizer component, the neutralizer component, the surfactant component, and the lubricant component are each present in the composition.

In some embodiments of the pharmaceutical compositions and processes described herein, the composition contains from about 0.5 mg to about 5.0 mg of active pharmacological agent, or from about 1.0 mg to about 3.0 mg of active pharmacological agent, or from about 1.5 mg to about 2.5 mg of active pharmacological agent, or from about 20 mg to about 30 mg of active pharmacological agent, or from about 22.5 mg to about 27.5 mg of active pharmacological agent, or from about 24.0 mg to about 26.0 mg of active pharmacological agent, or from about 50 mg to about 100 mg of active pharmacological agent, or from about 65 mg to about 85 mg of active pharmacological agent, or from about 70 mg to about 80 mg of active pharmacological agent, or from about 75 mg to about 125 mg of active pharmacological agent, or from about 90 mg to about 110 mg of active pharmacological agent, or from about 72 mg to about 76 mg of active pharmacological agent.

In some further embodiments, the composition contains from about 50.0 mg to about 750.0 mg of active pharmacological agent, or from about 50.0 mg to about 200.0 mg of active pharmacological agent, or from about 100.0 mg to about 175.0 mg of active pharmacological agent, or from about 125.0 mg to about 175.0 mg of active pharmacological agent.

In some further embodiments, the composition contains from about 100.0 mg to about 750.0 mg of active pharmacological agent, or from about 150.0 mg to about 750.0 mg of active pharmacological agent, or from about 200.0 mg to about 750.0 mg of active pharmacological agent, or from about 300.0 mg to about 750.0 mg of active pharmacological agent. In some embodiments, the composition contains about 2 mg, about 4 mg, about 10 mg, about 25 mg, about 50 mg, about 75 mg, about 100 mg, about 150 mg, about 200 mg, about 300 mg, about 400 mg, about 450 mg, about 500 mg, about 600 mg, about 700 mg, or about 750 mg of active pharmacological agent.

In some embodiments of each of the foregoing compositions, the composition comprises a plurality of enteric-coated pellets. In some such embodiments, the enteric-coated pellets are present in a capsule.

The present invention further provides processes for preparing the disclosed pharmaceutical compositions, comprising:

a) preparing uncoated pellets comprising said filler and said active pharmacological agent;

b) optionally applying a subcoat to the uncoated pellets; and

c) applying an enteric coating to the pellets.

In some embodiments, the processes further comprise the step of:

d) filling a capsule with said pellets to achieve a predetermined dose of Compound A, or a pharmaceutically acceptable salt thereof, preferably Compound A.HCl.

In some embodiments, step (a) includes:

i) mixing the filler and the active pharmacological agent to form a mixture;

ii) wet granulating the mixture to form a granulate; and

iii) extruding and spheronizing the granulate.

In some embodiments, step (c) includes:

i) preparing a suspension comprising said enteric coat, said plasticizer, said neutralizer, and said surfactant; and

ii) spraying said suspension onto said pellets.

The invention also provides products of the processes described herein.

In some embodiments, the invention provides pharmaceutical compositions comprising a plurality of enteric coated pellets containing a composition as described herein. In some embodiments, the pellets comprise:

a) an active pharmacological agent comprising from about 20% to about 22% by weight of the pharmaceutical composition;

b) a filler component comprising from about 60% to about 66% by weight of the pharmaceutical composition;

c) an optional seal coat component comprising, when present, from about 1% to about 2% by weight of the pharmaceutical composition;

d) an enteric coat component comprising from about 8% to about 12% by weight of the pharmaceutical composition;

e) an optional glidant component comprising, when present, from about 0.1% to about 0.3% by weight of the pharmaceutical composition;

f) an optional plasticizer component comprising, when present, from about 0.5% to about 1.0% by weight of the pharmaceutical composition;

g) an optional neutralizer component comprising, when present, from about 0.05% to about 0.3% by weight of the pharmaceutical composition;

h) an optional surfactant component comprising, when present, from about 0.005% to about 0.025% by weight of the pharmaceutical composition; and

i) an optional lubricant component comprising, when present, from about 2.5% to about 3.5% by weight of the pharmaceutical composition;

wherein:

the active pharmacological agent comprises Compound A, or a pharmaceutically acceptable salt thereof, preferably Compound A.HCl;

the filler component comprises microcrystalline cellulose;

the seal coat component, when present, comprises, Opadry® II Clear;

the enteric coat component comprises a methacrylic copolymer such as Eudragit® L30D-55;

the glidant component, when present, comprises mono- and di-glycerides;

the plasticizer component, when present, comprises triethyl citrate;

the neutralizer component, when present, comprises NaOH;

the surfactant component, when present, comprises polysorbate 80; and

the lubricant component, when present, comprises talc.

In some embodiments of the pharmaceutical compositions comprising a plurality of enteric coated pellets described above, the active pharmacological agent comprises from about 30% to about 80% by weight of the pharmaceutical composition, or from about 30% to about 45% by weight of the pharmaceutical composition, or from about 50% to about 70% by weight of the pharmaceutical composition, or from about 60% to about 70% by weight of the pharmaceutical composition; the filler component comprises from about 40% to about 60% by weight of the pharmaceutical composition, or from about 10% to about 30% by weight of the pharmaceutical composition; the optional seal coat component, when present, comprises from about 0.5% to about 3% by weight of the pharmaceutical composition; the enteric coat component comprises from about 5% to about 15% by weight of the pharmaceutical composition; the optional glidant component, when present, comprises from about 0.1% to about 2% by weight of the pharmaceutical composition; the optional plasticizer component, when present, comprises from about 0.1% to about 1.5% by weight of the pharmaceutical composition; the optional neutralizer component, when present, comprises from about 0.01% to about 0.8% by weight of the pharmaceutical composition; the optional surfactant component, when present, comprises from about 0.005% to about 0.05% by weight of the pharmaceutical composition; and the optional lubricant component, when present, comprises from about 1% to about 4% by weight of the pharmaceutical composition.

In some embodiments, each of said components (c), (e), (f), (g), (h) and (i) are present.

In some embodiments, and particularly in embodiments having less than about 30%, or less than about 25% of filler, it may be advantageous to further include a binder in the pellet, to facilitate pellet cohesion. Any of the many known binders for pharmaceutical formulations can be employed, for example and not limitation HPMC and polyvinylpyrrolidone. Further examples of suitable binders can be found in, for example, Remington's Pharmaceutical Sciences, infra. The binder can be present in any effective amount, for example up to about 5% by weight of the pharmaceutical composition.

In some embodiments of each of the pharmaceutical compositions described herein, the compositions can further include a sustained release component. Generally, the sustained release component is a coating that causes the sustained release of the active pharmacological agent. In some embodiments, the sustained release coating is disposed in between the optional seal coat component and the enteric coat component. Generally, the sustained release coating includes one or more release-controlling excipients. Any of the wide variety of such materials known in the art are suitable. Examples of suitable release-controlling excipients include one or more of gelatin, shellac, hydroxypropylmethyl cellulose (HPMC), methylcellulose (MC), ethylcellulose (EC), hydroxypropylmethyl cellulose phthalate (HPMCP), cellulose acetate phthalate (CAP), methacrylic acid/methyl methacrylate copolymers, polyvinyl acetate phthalate (PVAP), glyceryl behenate, paraffin or carnauba wax. Further examples of suitable release-controlling excipients can be found in Remington's Pharmaceutical Sciences, supra.

It will be understood that the weight percentages set forth for the components of the pharmaceutical compositions described herein are the percentages that each component will comprise of a final pharmaceutical composition, without reference to a capsule. In some embodiments, the compositions of the invention include or are composed of enteric-coated pellets. In some such embodiments, a desired dosage for the composition can be achieved by filling a capsule or like delivery vehicle with a desired amount of the enteric-coated pellets. In some instances where low dosages are desired, an additional filler can be added to the enteric-coated active pellets to increase the fill weight of the capsule. Any convenient filler useful in the pharmaceutical arts can be used. One non-limiting example of such an additional filler is inert sugar spheres.

Generally, the active pharmacological agent(s) can be present in from about 10% to about 80% by weight of the pharmaceutical composition. In some embodiments, the active pharmacological agent is present in from about 15% to about 25% by weight of the pharmaceutical composition, or from about 20% to about 22% by weight of the pharmaceutical composition, or from about 30% to about 40% by weight of the pharmaceutical composition. In some further embodiments, the active pharmacological agent is present in from about 30% to about 80% by weight of the pharmaceutical composition, or from about 30% to about 45% by weight of the pharmaceutical composition, or from about 50% to about 70% by weight of the pharmaceutical composition, or from about 60% to about 70% by weight of the pharmaceutical composition. In some embodiments, the active pharmacological agent comprises Compound A, or a pharmaceutically acceptable salt thereof.

The filler component generally comprises from about 10% to about 80% by weight, from about 50% to about 70% by weight, or from about 60% to about 66% by weight, or from about 40% to about 50% by weight, of the pharmaceutical composition. In some further embodiments, the filler component comprises from about 10% to about 30%, or from 10% to about 20% of the pharmaceutical composition. The filler component can include any filler compound useful for preparing pharmaceutical preparations. Examples of suitable fillers include one or more of microcrystalline cellulose, lactose, starch, carboxymethyl cellulose, cellulose gum, polyethylene glycol, other substituted celluloses, for example ethyl cellulose, carboxyethyl cellulose, hydroxyethyl celluloses, calcium phosphates such as anhydrous dicalcium phosphate, metal aluminosilicates, such as magnesium aluminometasilicate (e.g., Neusilin®), sugar or carbohydrate containing compounds such as mannitol, sucrose, maltodextrin, sorbitol, starch and xylitol, as well as metal phosphates and metal carbonates, for example magnesium carbonate. Other suitable filler materials can be found in, for example, Remington's Pharmaceutical Sciences, 17th ed., Mack Publishing Company, Easton, Pa., 1985, which is incorporated herein by reference in its entirety. In more preferred embodiments, the filler is microcrystalline cellulose.

Generally, the pharmaceutical compositions of the invention are delay release formulations. In some embodiments, delay release is achieved by an enteric coat component (a gastro-resistant coating), which prevents release to the active pharmacological agent until the small intestine. Thus, in some embodiments, the enteric coat component provides for release of the active pharmacological agent at a pH greater than that found in the stomach (i.e., a pH greater than about 1 to about 2), and preferably at a pH suitable to provide release of the active pharmacological agent in the small intestine (e.g., a pH of at least about 5; for example a pH of from about 5 to about 7).

In some embodiments, a seal coat (i.e., a subcoat) is optionally applied first, which provides a more uniform surface prior to enteric coating. In some embodiments, the seal coat is essentially an instant release coat; i.e., it does not significantly retard the release of the contents under the coating. A variety of suitable seal coats as are known in the art can be used. Non-limiting examples of such seal coats include one or more of Opadry® II Clear, other Opadry® coat materials (available from Colorcon), Kollicoat® (IR, IR White or Protect grades, available from BASF), maltodextrin and Pure-Cote® (available from Grain Processing Corp., Muscatine, Iowa), Pharmacoat® (a hydroxypropylmethyl cellulose type polymer available from Shin-Etsu Chemical Co., Ltd., New York, N.Y.), and other cellulose- or starch-base coats. Further examples can be found in Remington's Pharmaceutical Sciences, supra. In some preferred embodiments, the seal coat component includes Opadry® II clear. Generally, the optional seal coat component, when present, comprises from about 0.01% to about 5%, from about 0.5% to about 3%, or from about 1% to about 2% by weight, or from about 2% to about 3% by weight, of the pharmaceutical composition.

Generally, the enteric coat component comprises from about 0.01% to about 20% by weight, or from about 5% to about 15% by weight, or from about 8% to about 12% by weight, or from about 12% to about 16% by weight, or from about 9% to about 11% by weight, or from about 9.6% to about 10.6% by weight, or from about 10.0% to about 10.2% by weight, of the pharmaceutical composition. The enteric coat component can include any enteric coating useful in the pharmaceutical arts.

Typically, enteric coating contain one or more polymers that serve to impart the enteric release characteristics of the coating. Examples of suitable enteric coat components (gastro-resistant coatings) include cellulose acetate phthalate (CAP) (e.g., Aquacoat® CPD), co-processed polyvinyl acetate phthalate (PVAP) (e.g., Suretetic), cellulose acetate trimellitate (CAT), methacrylic copolymers (such as Eudragit® type polymers from Röhm America, LLC, Piscataway, N.J., a subsidiary of Röhm GmbH & Co. KG, Germany, a division of Degussa AG) such as Eudragit® L30D-55 dry polymer, which is a dispersion of methyacrylic copolymers with an anionic functional group at a pH that is carboxylic acid (30% solids), Acryl-EZE® (Colorcon, West Point, Pa.), which is a methacrylic acid co-polymer type C, Eastacryl® (Eastman Chemical Company, Kingsport, Tenn.), which is an aqueous dispersion of acrylic polymer, hydroxypropyl methylcellulose phthalate (HPMCP), hydroxypropyl methyl cellulose acetate succinate (HPMCAS), and other HPMC containing enteric coatings, such as Spectrablend™ (Sensient Pharmaceutical Technologies, South Plainfield, N.J.). Further examples can be found in Remington's Pharmaceutical Sciences, supra. In some more preferred embodiments, the enteric coat component includes Eudragit® L30D-55 dry polymer. In some embodiments, the coating is applied such that a weight gain of enteric polymer (e.g., Eudragit® L30D-55 dry polymer) of from about 12% to about 22% is achieved, relative to the weight of the uncoated pellet. Generally, the optional glidant component, when present, comprises from about 0.01% to about 2% by weight, about 0.01% to about 1% by weight, or from about 0.1% to about 0.3% by weight, or from about 0.1% to about 3% by weight, of the pharmaceutical composition. The glidant can be any of those known in the art. Non-limiting examples include one or more of mono- and di-glycerides, talc, silicon dioxide, stearic acid, starch, powdered cellulose, lactose, stearates, calcium phosphates, magnesium carbonate, magnesium oxide, silicates, and silicon dioxide aerogels. Further examples can be found in Remington's Pharmaceutical Sciences, supra. In some preferred embodiments, the glidant includes mono- and di-glycerides, for example Imwitor® 900K.

Generally, the optional plasticizer component, when present, comprises from about 0.01% to about 3% by weight, from about 0.1% to about 1.5% by weight, or from about 0.5% to about 1.0% by weight, or from about 0.5% to about 2.0% by weight, of the pharmaceutical composition. A variety of plasticizers are useful in the compositions described herein. Non-limiting examples include one or more of triethyl citrate, dibutyl sebecate, polyethylene glycol, propylene glycol, triacetin, sorbitol, tributyl citrate, acetyltributyl citrate, acetyltriethyl citrate, dibutyl phthalate, triethyl citrate and triethanolamine. Further examples of suitable plasticizers can be found in Remington's Pharmaceutical Sciences, supra. In some preferred embodiments, the plasticizer component includes triethyl citrate.

Generally, the optional neutralizer component, when present, comprises from about 0.01% to about 1.5%, from about 0.01% to about 0.8%, or from about 0.05% to about 0.3% by weight, or from about 0.003% to about 0.3% by weight, of the pharmaceutical composition. Any neutralizing compound suitable for the pharmaceutical arts can be used. Non-limiting examples include one or more of NaOH, KOH and NH₄OH. Further examples of suitable surfactants can be found in Remington's Pharmaceutical Sciences, supra. In some preferred embodiments, the neutralizer component includes NaOH.

Generally, the optional surfactant component, when present, comprises from about 0.001% to about 1.0%, 0.005% to about 0.05% by weight, or from about 0.005% to about 0.025% by weight, or from about 0.001% to about 0.3% by weight, of the pharmaceutical composition. A variety of surfactants as know in the art can be used. Non-limiting examples include one or more of polysorbate 80, sodium lauryl sulfate, sucrose palmitate, poloxamer, docusate sodium, and polyoxyethylene sorbitan fatty acid esters, polyoxyethylene stearates, sucrose fatty acid esters and sorbitan fatty acid esters. Further examples of suitable surfactants can be found in Remington's Pharmaceutical Sciences, supra. In some preferred embodiments, the surfactant component includes polysorbate 80.

Generally, the optional lubricant component comprises from about 0.01% to about 5.0% by weight, from about 1% to about 4% by weight, or from about 2.5% to about 3.5% by weight, or from about 2.0% to about 3.5% by weight, of the pharmaceutical composition. A variety of lubricants as are known in the art can be used in the present compositions. Non-limiting examples of suitable lubricants include one or more of talc, metallic stearates, silicon dioxide, sodium stearyl fumarate, fatty acid esters, fatty acids, fatty alcohols, glyceryl behenate, mineral oil, paraffins, hydrogenated vegetable oils, leucine, polyethylene glycols, metallic lauryl sulfates, silica such as Aerosil® 200, and sodium chloride. Further examples of suitable lubricants can be found in Remington's Pharmaceutical Sciences, supra. In some preferred embodiments, the lubricant component includes talc.

The formulations described herein can include any conventionally used oral forms, including, for example, tablets, pellets, capsules, buccal forms, troches, lozenges and oral liquids, suspensions, and the like. Capsules are preferred. Capsules or tablets containing the present formulation can also be combined with mixtures of other active compounds or inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In some preferred embodiments, the formulations are contained in capsules.

Tablet formulations can be made by conventional compression methods and utilize pharmaceutically acceptable diluents or fillers, binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar.

Additional film coatings useful with the present formulations are known in the art and generally consist of a polymer (usually a cellulosic type of polymer), a colorant and a plasticizer. Additional ingredients such as wetting agents, sugars, flavors, oils and lubricants can be included in film coating formulations to impart certain characteristics to the film coat. The compositions and formulations herein may also be combined and processed as a solid, then placed in a capsule form, such as a gelatin capsule.

As will be appreciated, some components of the formulations of the invention can possess multiple functions. In some such cases, the function of a given component can be considered singular, even though its properties may allow multiple functionality.

The pharmaceutical compositions and excipient systems herein can also contain an antioxidant or a mixture of antioxidants, such as ascorbic acid. Other antioxidants which can be used include sodium ascorbate and ascorbyl palmitate, optionally in conjunction with an amount of ascorbic acid. An example range for the antioxidant(s) is from about up to about 15% by weight, e.g., from about 0.05% to about 15% by weight, from about 0.5% to about 15% by weight, or from about 0.5% to about 5% by weight. In some embodiments, the pharmaceutical compositions contain substantially no antioxidant.

Additional numerous various excipients, dosage forms, dispersing agents and the like that are suitable for use in connection with the solid dispersions of the invention are known in the art and described in, for example, Remington's Pharmaceutical Sciences, supra.

In some embodiments, the pharmaceutical compositions containing Compound A.HCl, and dosage forms including the same, described herein, provide therapeutically efficacious amounts of Compound A.HCl to patients in need thereof. It will be recognized by those of skill in the art that such dosages can be provided by a single or unit dosage form of the invention, or by administration of multiple such dosage forms. For example, in some embodiments, patients are treated with a total 24 hour dosage of from about 350 mg to about 400 mg of Compound A.HCl, preferably given in two dosages of from about 175 mg to about 200 mg, BID. Such dosages can be achieved by administering a single dosage form of the invention, i.e., a dosage form containing or consisting of a composition of the invention, that contains the desired dose of Compound A.HCl. Alternatively, a plurality of such dosage forms that together contain the desired dosage, can be used. For example, two dosage forms of the invention containing 100 mg each, can be used to achieve a 200 mg dose of Compound A.HCl.

In some embodiments, the formulations of the invention provide enteric release of active pharmacological agent, preferably Compound A.HCl. In some embodiments, the release of Compound A.HCl is effective to provide:

a mean plasma concentration profile for Compound A.HCl in human schizophrenia patients which has a mean AUCss of about 33.23 hr*ng/mL±20% for a dosage of 100 mg Compound A.HCl, or a respective mean AUC value about proportional thereto for a total dose other than 100 mg; or a mean plasma concentration profile for Compound A.HCl in human schizophrenia patients which has a mean AUCss of about 54.88 hr*ng/mL±20% for a dosage of 150 mg Compound A.HCl, or a respective mean AUC value about proportional thereto for a total dose other than 150 mg; or a mean plasma concentration profile for Compound A.HCl in human schizophrenia patients which has a mean AUCss of about 173.49 hr*ng/mL±20% for a dosage of 250 mg Compound A.HCl, or a respective mean AUC value about proportional thereto for a total dose other than 250 mg; or
a mean plasma concentration profile for Compound A.HCl in healthy humans which has a mean AUCss of about 8.73 hr*ng/mL±20% for a dosage of 25 mg Compound A.HCl, or a respective mean AUC value about proportional thereto for a total dose other than 25 mg; or
a mean plasma concentration profile for Compound A.HCl in healthy humans which has a mean AUCss of about 26.49 hr*ng/mL±20% for a dosage of 50 mg Compound A.HCl, or a respective mean AUC value about proportional thereto for a total dose other than 50 mg; or
a mean plasma concentration profile for Compound A.HCl in healthy humans which has a mean AUCss of about 24.95 hr*ng/mL±20% for a dosage of 75 mg Compound A.HCl, or a respective mean AUC value about proportional thereto for a total dose other than 75 mg; or
a mean plasma concentration profile for Compound A.HCl in healthy humans which has a mean AUCss of about 62.76 hr*ng/mL±20% for a dosage of 100 mg Compound A.HCl, or a respective mean AUC value about proportional thereto for a total dose other than 100 mg; or
a mean plasma concentration profile for Compound A.HCl in healthy humans which has a mean AUCss of about 109.50 hr*ng/mL±20% for a dosage of 150 mg Compound A.HCl, or a respective mean AUC value about proportional thereto for a total dose other than 150 mg; or
a mean plasma concentration profile for Compound A.HCl in healthy humans which has a mean AUCss of about 234.75 hr*ng/mL±20% for a dosage of 250 mg Compound A.HCl, or a respective mean AUC value about proportional thereto for a total dose other than 250 mg; or

a Tₘₐₓ of from about 4 hours to about 6.5 hours in a in healthy humans or in a schizophrenia patient.

In some further embodiments, the invention provides unit dosage forms comprising from about 2 to about 150 mg of Compound A.HCl, the dosage form providing a Cₘₐₓ of Compound A between about 4 and about 8 hours after administration to a subject.

In some further embodiments, the invention provides unit dosage forms of a medicament, comprising Compound A.HCl; and a degradable coating, characterized in that the coating degrades so that less than 30% of the Compound A is released after two hours.

In some further embodiments, the invention provides unit dosage forms of a medicament having a uniform dosage of Compound A.HCl, the dosage form being characterized by a dissolution profile upon oral administration in which the Compound A.HCl is released such that a Cₘₐₓ of Compound A occurs between about 4 and about 8 hours after administration to a subject. In some such embodiments, the dosage form comprises a plurality of enterically coated pellets.

In some further embodiments, the invention provides a coated extrudate comprising Compound A.HCl and a binder or filler, characterized in that, when formulated into a unit dosage form, achieves release of Compound A.HCl, such that a Cₘₐₓ of Compound A occurs between about 4 and about 8 hours after administration to a subject. In some such embodiments, the filler or binder is selected from the group consisting of comprises one or more of microcrystalline cellulose, lactose, starch, carboxymethyl cellulose, cellulose gum, polyethylene glycol, substituted celluloses, ethyl cellulose, carboxyethyl cellulose, hydroxyethyl celluloses, calcium phosphates, anhydrous dicalcium phosphate, metal aluminosilicates, magnesium aluminometasilicate (Neusilin®), sugar or carbohydrate containing compounds, mannitol, sucrose, maltodextrin, sorbitol, starch, xylitol, metal phosphates, metal carbonates, and magnesium carbonate, preferably microcrystalline cellulose.

In some further embodiments, the invention provides methods of preparing a formulation comprising Compound A.HCl, the method comprising steps of:

providing pellets comprising Compound A.HCl; and

applying to the pellets an enteric coating comprising an enteric coating polymer, in an amount that provides a weight gain of enteric coating polymer of from about 12% to about 22% relative to the weight of the uncoated pellet.

In some further embodiments, the invention provides methods of preparing a formulation comprising Compound A.HCl, the method comprising steps of:

providing pellets comprising Compound A.HCl; and

applying to the pellets an enteric coating, wherein the coating degrades after administration of the formulation, such that the Compound A.HCl is released such that a Cₘₐₓ of Compound A occurs between about 4 and about 8 hours after administration to a subject. In some such embodiments, the coating achieves a dissolution profile characterized by less than 30% release of Compound A.HCl after 2 hours.

As discussed below, upon administration of formulations of the invention, a minor secondary peak was seen at ˜24-28 hrs after administration at steady state in most of the subjects at all dose groups. Accordingly, in some embodiments, the formulations of the invention provide blood serum levels of Compound A.HCl that are characterized by a maximum peak, followed by a secondary peak of lesser value.

“Cₘₐₓ,” “Tₘₐₓ,” and “AUC” values reported herein, unless stated as being “mean” values, refer to the values observed in an individual patient. Moreover, Cₘₐₓ, Tₘₐₓ, and AUC values, unless otherwise stated, may be values observed at steady state when dosing at regular time intervals (e.g., every 12 hours) for multiple days (e.g., multiple dose administration) or values for a single dose administration. In some embodiments, the formulations of the invention provide release characteristics for active pharmacological agents, e.g., Compound A.HCl, that conform with one or more of the kinetic parameters described below, for example Cₘₐₓ, Tₘₐₓ, and AUC. It is intended that such embodiments of the invention include values for these parameters that are ±20% of the values described in the Tables and Figures herein.

Alkyl, as used herein, refers to an aliphatic hydrocarbon chain and includes, but is not limited to, straight and branched chains such as methyl, ethyl, n-propyl, isopropyl, n-butyl, isobutyl, sec-butyl, t-butyl, n-pentyl, isopentyl, neo-pentyl, n-hexyl, and isohexyl. Lower alkyl refers to alkyl having 1 to 3 carbon atoms.

Alkanamido, as used herein, refers to the group R—C(═O)—NH— where R is an alkyl group of 1 to 5 carbon atoms.

Alkanol, as used herein, refers to the group R—C(═O)— where R is an alkyl group of 1 to 5 carbon atoms.

Alkanoyloxy, as used herein, refers to the group R—C(═O)—O— where R is an alkyl group of 1 to 5 carbon atoms.

Alkanesulfonamido, as used herein, refers to the group R—S(O)₂—NH— where R is an alkyl group of 1 to 6 carbon atoms.

Alkanesulfonyl, as used herein, refers to the group R—S(O)₂— where R is an alkyl group of 1 to 6 carbon atoms.

Alkoxy, as used herein, refers to the group R—O— where R is an alkyl group of 1 to 6 carbon atoms.

Aryl, as used herein, refers to an aromatic 5-to 7-membered monocarbocyclic ring such as phenyl. Heteroaryl means an aromatic 5-to 7-membered carbon containing monocyclic ring having one to two heteroatoms which independently may be nitrogen, oxygen or sulfur.

Aroyl, as used herein, refers to the group Ar—C(═O)— where Ar is aryl as defined above. For example, a C₆ to C₈ aroyl moiety refers to the group Ar—C (═O)— where Ar is an aromatic 5 to 7 membered carbocylic ring.

Alkylaryl, as used herein refers to the group —R—Ar where Ar is aryl as defined above and R is an alkyl moiety having 1 to 6, preferably 1 to 4, and more preferably 1 to 3 carbon atoms. Examples of alkylaryl groups include benzyl, phenethyl, 3-phenylpropyl, and 4-phenyl butyl. Alkylheteroaryl, as used herein, refers to the group-R-hetAr where hetar is heteroaryl as defined above and R is an alkyl moiety having 1 to 6, preferably 1 to 4, and more preferably 1 to 3 carbon atoms.

Carboxamido, as used herein, refers to the group NH₂—C(═O)—.

Carboalkoxy, as used herein, refers to the group R—O—C(═O)— where R is an alkyl group of 1 to 5 carbon atoms.

Carboarylalkoxy as used herein, refers to the group Ar—Ra—O—C(═O)— where Ar is aryl as defined above, and Ra is a lower alkyl group of 1 to 3 carbon atoms.

The pharmaceutical compositions of this invention may be used for the treatment of mental disorders, including psychotic disorders such as schizophrenia including paranoid type, disorganized type, catatonic type, and undifferentiated type, schizophreniform disorder, schizoaffective disorder, delusional disorder, substance-induced psychotic disorder, and psychotic disorder not otherwise specified; L-DOPA-induced psychosis; psychosis associated with Alzheimer's dementia; psychosis associated with Parkinson's disease; psychosis associated with Lewy body disease; bipolar disorders such as bipolar I disorder, bipolar II disorder, and cyclothymic disorder; depressive disorders such as major depressive disorder, dysthymic disorder, substance-induced mood disorder, and depressive disorder not otherwise specified; mood episodes such as major depressive episode, manic episode, mixed episode, and hypomanic episode; anxiety disorders such as panic attack, agoraphobia, panic disorder, specific phobia, social phobia, obsessive compulsive disorder, posttraumatic stress disorder, acute stress disorder, generalized anxiety disorder, separation anxiety disorder, substance-induced anxiety disorder, and anxiety disorder not otherwise specified; adjustment disorders such as adjustment disorders with anxiety and/or depressed mood; intellectual deficit disorders such as dementia, Alzheimer's disease, and memory deficit; eating disorders (e.g., hyperphagia, bulimia or anorexia nervosa) and combinations of these mental disorders that may be present in a mammal. For example, mood disorders such as depressive disorders or bipolar disorders often accompany psychotic disorders such as schizophrenia. A more complete description of the aforementioned mental disorders can be found in the Diagnostic and Statistical Manual of Mental Disorders, 4th edition, Washington, D.C., American Psychiatric Association (1994).

The pharmaceutical compositions of the present invention are also of interest for the treatment of epilepsy; migraines; sexual dysfunction; sleep disorders; gastrointestinal disorders, such as malfunction of gastrointestinal motility; and obesity, with its consequent comorbidities including Type II diabetes, cardiovascular disease, hypertension, hyperlipidemia, stroke, osteoarthritis, sleep apnea, gall bladder disease, gout, some cancers, some infertility, and early mortality. The pharmaceutical compositions of the present invention can also be used to treat central nervous system deficiencies associated, for example, with trauma, stroke, and spinal cord injuries. The pharmaceutical compositions of the present invention can therefore be used to improve or inhibit further degradation of central nervous system activity during or following the malady or trauma in question. Included in these improvements are maintenance or improvement in motor and motility skills, control, coordination and strength.

Thus the present invention provides methods of treating each of the maladies listed above in a mammal, preferably in a human, the methods comprising providing a therapeutically effective amount of a pharmaceutical composition of this invention to the mammal in need thereof. By “treating”, as used herein, it is meant partially or completely alleviating, inhibiting, preventing, ameliorating and/or relieving the disorder. For example, “treating” as used herein includes partially or completely alleviating, inhibiting or relieving the condition in question.

The materials, methods, and examples presented herein are intended to be illustrative, and are not intended to limit the scope of the invention. All publications, patent applications, patents, and other references mentioned herein are incorporated by reference in their entirety.

### EXAMPLES

As will be appreciated, the optimal amounts of the various constituents of the compositions disclosed herein can vary with the process parameters chosen for preparation of the compositions. The examples provided herein are not intended to limit the invention in any respect.

### Example 1

### Preparation of Enteric Coated Capsules Containing Compound A.HCl

High-dose capsules were prepared by making a 97.75% granulation of Compound A.HCl containing 2% Crospovidone and 0.25% Mg Stearate. Low-strength capsules were prepared by adding Lactose as a diluent. The granulations were made by the wet granulation process. Capsules were coated in a coating pan using a Clear Opadry® subcoat followed by an aqueous dispersion of Eudragit®. The coating was applied at a bed temperature of approximately 30-35° C. until the % weight gain of Eudragit® polymer was sufficient to prevent release of the drug substance in acid and yield complete release in approximately 60 min in pH 6.8 buffer. Small batches were prepared by hand using a solvent.

### Example 2

### Preparation of capsules containing compound A.HCl Enteric-Coated Pellets

These formulations consisted of spheres (pellets) containing Compound A.HCl that contained a subcoat, and an enteric coat. The three capsule strengths were prepared from a common stock of the enteric-coated pellets (approximately 20% w/w Compound A.HCl) by adjusting fill weights. Inert sugar spheres are added to the enteric-coated active pellets for the 2 mg capsules in order to bring the total fill weight into the range of the 25 and 75 mg capsule fill weight.

Preparation of Compound A.HCl Enteric Coated Pellets

Compound A.HCl 25% uncoated pellets were prepared by using the extrusion/spheronization process. The Compound A.HCl 25% uncoated pellets consisted of 25% Compound A.HCl drug substance and 75% Microcrystalline Cellulose (MCC). USP water was used to make wet granulation of 25% Compound A.HCl with MCC. The extrusion and spheronization processes below were used to prepare the cores.

The Compound A.HCl 25% uncoated pellets were prepared according to the following procedure:

1. Microcrystalline Cellulose (75%) and Compound A.HCl (25%) were mixed for 5 minutes.
2. The mix was granulated with purified water USP to form a wet mass suitable for extrusion.
3. The granulation was extruded using a Nica™ Extruder/spheronizer with a 1.0 mm screen.
4. The extrudate was spheronized using the Nica™ Extruder/spheronizer. Approximate spheronization time was 5 minutes.
5. The pellets were dried in a fluid bed dryer with an inlet temperature of 50° C.±5° C. to a moisture content of 3-4% tested on a Computrac at 100° C.

A representative particle size distribution of Compound A.HCl 25% uncoated pellets prepared by the procedure above is shown in Table 1 below:

TABLE 1

| | % w/w |
|---|---|
| Pellets >1000μ | 3.3 |
| Pellets <600μ | 7.5 |
| Pellets within size fraction 600-1000μ | 85.0 |
| Losses during processing | 4.2 |

Preparation of Compound A.HCl 20% Enteric Coated Pellets

The enteric coating level of Compound A.HCl 25% pellets was chosen from different coating levels which gave full protection from acid and has the least coating level. It was determined that in the enteric coating layer, 14% of dry Eudragit® polymer weight gain could provide full protection of the drug from acid, and then release of drug in buffer phase, with almost 100% of the drug being released after 2 hours in pH 6.8 buffer.

In-process dissolution testing was used to ensure that sufficient coating was applied to the pellets to provide protection from acid dissolution and complete release in pH 6.8 buffer.

1) Subcoating of Uncoated Pellets

A subcoat was applied onto active pellets using Opadray II Clear before applying enteric coating layer. A subcoat was used to give a uniform surface to the pellets prior to enteric coating. The Eudragit® dispersion was formulated with Glyceryl Monostearate (Imwitor®) to provide good lubrication to the dispersion without the use of talc, which tends to clog spray nozzles and be non-uniform in the dispersion. The following formulation was prepared:

Subcoat Coating Solution (5.0% wt/wt solid)

The 5.0% wt/wt of Opadry® II Clear Y-19-7483 solution was used in the Initial Seal Coat. The preparation of 1 kg of Seal Coating solution is shown below:

| INGREDIENT | AMOUNT |
|---|---|
| Opadry ® II Clear Y-19-7483 | 50.00 g |
| Purified Water, USP/BP/EP | 950.00 g |

Procedure:
1. Purified Water, USP/BP/EP, was placed in a suitable vessel equipped with a low shear (Lightnin type) or suitable mixer.
2. Opadry® II Clear, Y-19-7483, was added in small increments to the vortex created in the Purified Water, USP/BP/EP by the moderate agitation in Step 1.
3. Agitation of the Step 2 mixture was continued for a minimum of one hour or until all the Opadry® II Clear wais dissolved.
4. The Step 3 solution was brought to the total theoretical weight with Purified Water, USP/BP/EP, if necessary.
5. The solution was stored in a well-closed container at room temperature awaiting use. The solution was used within 24 hours when stored at room temperature. The solution may be stored at 2° C.-10° C. for 84 hours.

The following Coating Parameters were used for the initial seal coating:

| | Target |
|---|---|
| Inlet Air Temperature (° C.) | 42 |
| Exhaust Air Temperature (° C.) | 32 |
| Air Volume (M³/H) | 70 |
| Atomizing Air (bar) | 1-1.5 |
| Spray Rate (mL/min) | 8-10 |
| Final Exhaust Drying Air temperature (° C.) | 42 |

2) Application of Enteric Coat to Pellets

After the subcoat, a layer of approximate 14% enteric coating (14.81% wt/wt solid based on dry polymer weight) was applied on the pellets. The coating solution formulation and coating parameters are listed below:

Preparation of Enteric Coating Dispersion (14.81% wt/wt solid)

A. Imwitor® 900K Dispersion Preparation

The formula for 200 g of Imwitor® 900K dispersion is as follows:

| INGREDIENT | AMOUNT | |
|---|---|---|
| Imwitor ® 900K | 20.00 | g |
| Triethyl Citrate | 20.00 | g |
| Tween 80, Vegetable grade | 0.80 | g |
| Purified Water, USP/BP/EP q.s. to | 200.00 | g |

Procedure:
1. Purified Water, USP/BP/EP, Imwitor® 900K, Triethyl Citrate and Tween 80, Vegetable grade, were poured into a suitable size beaker and heated up to 65° C. with agitation by magnetic stir bar.
2. The mixture was homogenized for 15 mins by using a suitable homogenizer.
3. After cooling down to 20° C., this mixture was added to a polymer dispersion.
   B. Enteric Coating Suspension Preparation

The following shows the formula for 1 kg of Enteric Coating Suspension:

| INGREDIENT | AMOUNT | |
|---|---|---|
| Eudragit ® L30D-55 (30% wt/wt solids) dispersion | 444.44 | g |
| Triethyl citrate, NF | 6.67 | g |
| Imwitor ® 900K dispersion from Step A | 26.67 | g |
| Sodium hydroxide, NF 1 N solution | 55.56 | g |
| Purified Water, USP/BP/EP q.s. to | 1000.00 | g |

Procedure:
1. Eudragit® L30D-55 was placed in a suitable vessel equipped with a low shear (Lightnin type) or suitable mixer. Note: Eudragit® should be screened through a 180 micron hand screen prior dispensing.
2. With moderate agitation, Triethyl citrate, NF and Imwitor® 900K dispersion was gradually added to the Step 1 suspension. Then 400.00 g of Purified Water was added to the suspension and agitation was continued for a minimum of 60 minutes.
3. Sodium hydroxide, NF 1 N solution was added to the suspension from Step 2 gradually to adjust the pH to 5.45.
4. The Step 3 suspension was brought to the total theoretical weight with Purified Water, USP/BP/EP, if necessary, and mixed for a minimum of 30 minutes to achieve a complete uniform mixture.
5. The mixture was stored under agitation at room temperature up to 48 hours before starting the spraying process.
6. The mixture was screened through a 180 micron hand screen before use.
   Coating Parameters for Enteric Coating

The following shows target coating parameters for the enteric coating process:

| | Target |
|---|---|
| Inlet Air Temperature (° C.) | 32 |
| Exhaust Air Temperature (° C.) | 25 |
| Air Volume (M³/H) | 70 |
| Atomizing Air (bar) | 1-1.5 |
| Spray Rate (mL/min) | 8-10 |
| Final Exhaust Drying Air temperature (° C.) | 30 |

The formulation of the Compound A.HCl 2, 25, 75 and 100 mg enteric coated pellets are shown below in Table 2:

TABLE 2

| | 2 mg | 25 mg | 75 mg | 100 mg |
|---|---|---|---|---|
| Pellet Ingredients | strength | strength | strength | strength |
| Compound A HCl | 2.0 | 25.0 | 75.0 | 100.0 |
| MCC | 6.04 | 75.0 | 225.0 | 122.2 |
| Purified Water | — | — | — | — |
| Seal Coat | | | | |
| Opadry ® II Clear | 0.16 | 2.0 | 6.0 | 6.7 |
| Purified Water | — | — | — | — |
| Enteric Coat | | | | |
| Eudragit ® L30D-55 | 0.966 | 12.0 | 36.0 | 40.0 |
| dry polymer | | | | |
| Mono- and Di- | 0.019 | 0.24 | 0.72 | 4.01 |
| Glycerides | | | | |
| Triethyl Citrate | 0.068 | 0.84 | 2.52 | 2.98 |
| NaOH | 0.016 | 0.20 | 0.60 | 0.21 |
| Polysorbate 80 | 0.0008 | 0.0096 | 0.0288 | 0.40 |
| Purified Water | — | — | — | — |
| Talc | 0.279 | 3.46 | 10.38 | 7.70 |
| Total Weight (mg) | 9.56 | 118.75 | 356.25 | 284.2 |
| Encapsulation of | | | | |
| Enteric Pellets | | | | |
| Sugar Spheres | 240.44 | — | — | — |
| Total Fill Weight | 250.0 | 118.75 | 356.25 | 284.2 |
| (mg) | | | | |

The function of excipients used in the formulation of Compound A.HCl 2, 25, 75 and 100 mg enteric coated pellets are listed in Table 3, below.

TABLE 3

| Ingredient | % Wt/Wt | Functionality |
|---|---|---|
| Compound A HCl | 21.05 | Active |
| MCC | 63.16 | Filler |
| Opadry ® II Clear | 1.68 | Seal Coat |
| Eudragit ® L30D-55 Dry | 10.11 | Enteric Coat |
| Polymer | | |
| Mono- and Di-Glycerides | 0.20 | Glidant |
| Triethyl Citrate | 0.71 | Plasticizer |
| NaOH | 0.17 | Neutralizer |
| Polysorbate 80 | 0.01 | Surfactant |
| Talc | 2.91 | Lubricant |
| Purified Water | Qs | Vehicle |

### Example 3

### Preparation of Capsules Containing 75, 100, 125 AND 150 mg Strength Compound A.HCl Enteric-Coated Pellets

The procedure of Example 2 was used, with the difference that the uncoated pellets contained 45% Compound A.HCl, and 55% Microcrystalline Cellulose (MCC). The coating of the pellets was performed as in Example 2 (the same formulation applied to the same weight gain), but containing 1% mono- and di-glycerides. Capsules were then filled to a dosage of 150 mg. If desired, capsules can be filled to other dosages, for example 75 mg, 100 mg, or 125 mg.

### Example 4

Preparation of Capsules Containing 200, 300, 500 and 750 mg Strength Compound A.HCl Enteric-Coated Pellets

The procedure of Example 2 is used, with the difference that the uncoated pellets contain 80% Compound A.HCl, and 20% Microcrystalline Cellulose (MCC). The coating of the pellets is performed as in Example 2 (the same formulation applied to the same weight gain), but containing 1% mono- and di-glycerides. Capsules are then filled to dosages of 200 mg, 300 mg, 500 mg or 750 mg.

### Example 5

Comparison of Oral Solution and Enteric Formulations

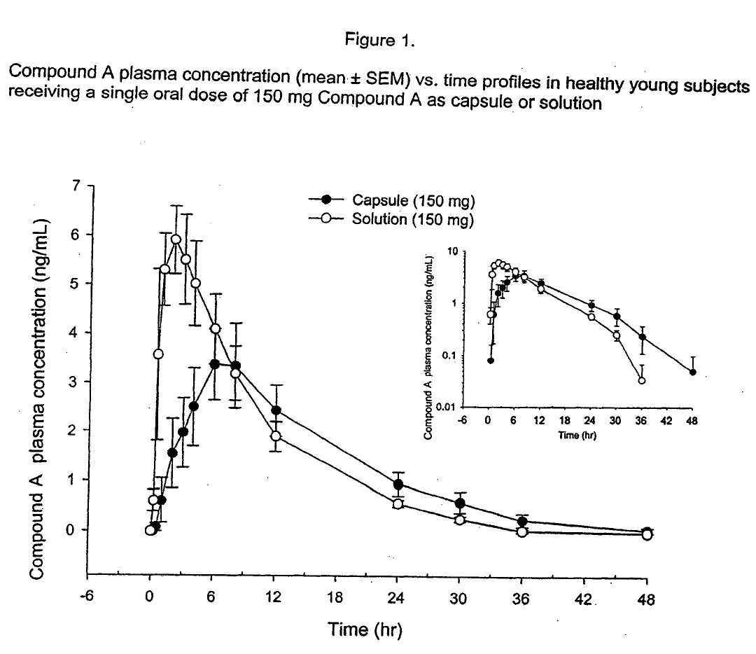

Compound A (150 mg) was administered to six healthy young subjects in the form of an oral solution and as 150 mg enteric capsules, after an overnight fast of 10 hours. Upon oral administration, Compound A was absorbed more rapidly from the solution in comparison to the enteric-coated capsule formulation (150 mg, fasted) with a mean Tₘₐₓ of 2.3 hours (vs. mean Tₘₐₓ=7.0 hours for capsule). As can be seen from FIG. 1, Compound A Cₘₐₓ was approximately 1.8 fold higher with the solution in comparison to the capsule (mean Cₘₐₓ with solution=7.5 ng/mL vs. mean Cₘₐₓ for capsule=4.1 ng/mL, p<0.05). Elimination t_{1/2} of Compound A was found to be approximately 6.6 hours. There was no significant difference in Compound A AUC, CL/F, Vz/F and t_{1/2} with the 2 different formulations (p>0.05).

These results show that the although the Cₘₐₓ for the enteric capsules was less, therefore providing a reduced potential for GI side effects, the bioavailabilty of the two formulations is equivalent. In conclusion, the solution does not behave significantly different from the capsule in terms of exposure and clearance.

### Example 6

### Administration of Enteric Coated Capsules Containing Compound A.HCl to Healthy Subjects

Ascending multiple doses of 25, 50, 75, 100, 150, and 250 mg Compound A.HCl were administered as twice-daily regimen (q12h) for 14 days in cohorts of 8 healthy subjects (6 active and 2 placebo). Blood samples for Compound A.HCl analysis were obtained on days 1 and 14 within 2 hours of test article administration and at various time points from 0.5 to 48 hours after test article administration. Doses were administered orally after an overnight fast of at least 10 hours on these 2 days. Trough PK samples were collected on days 7, 10, and 12 to assess whether steady-state had been reached.

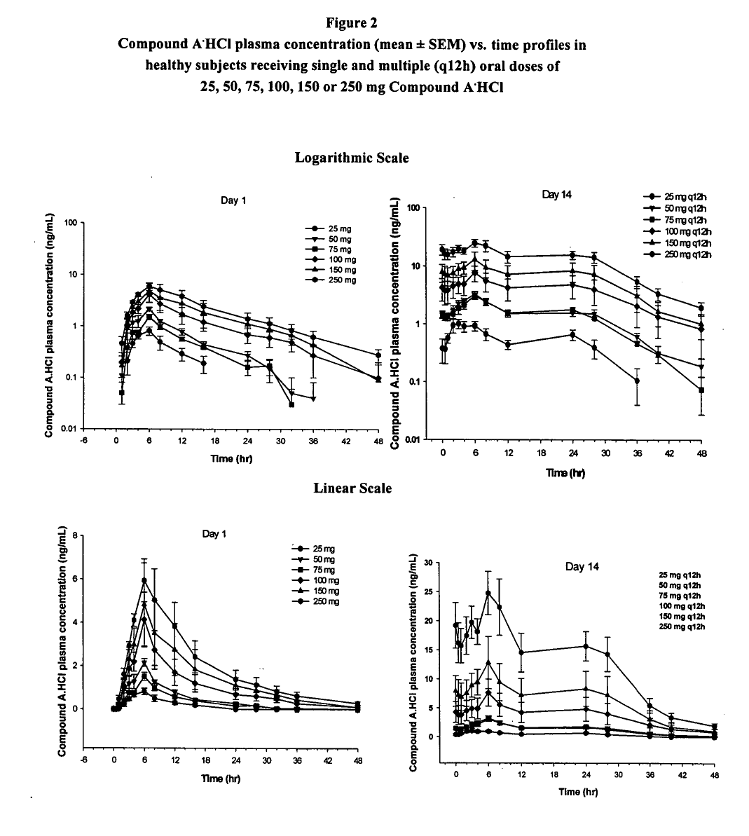

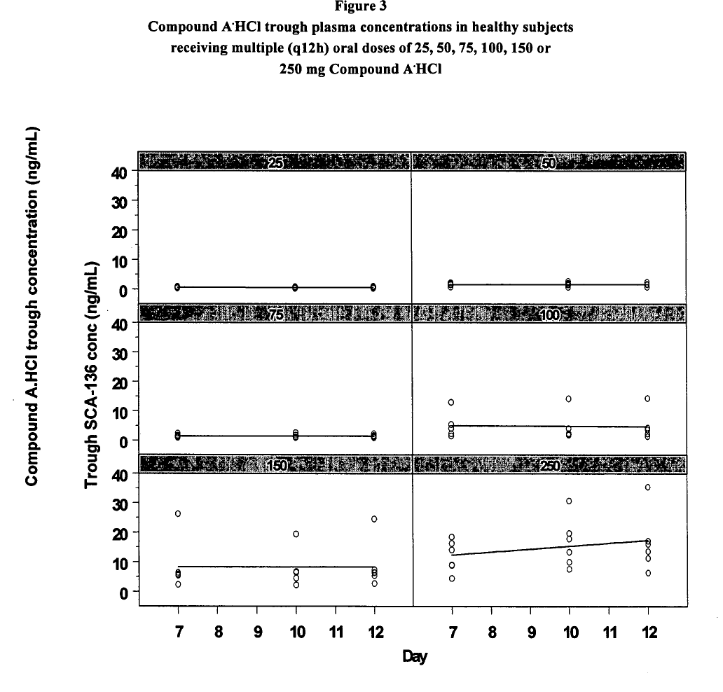

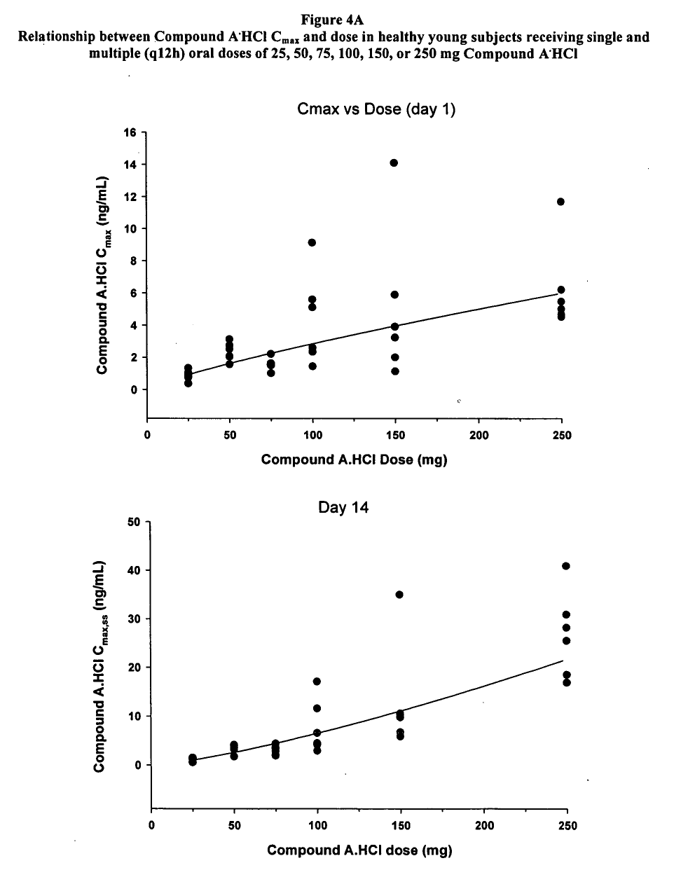

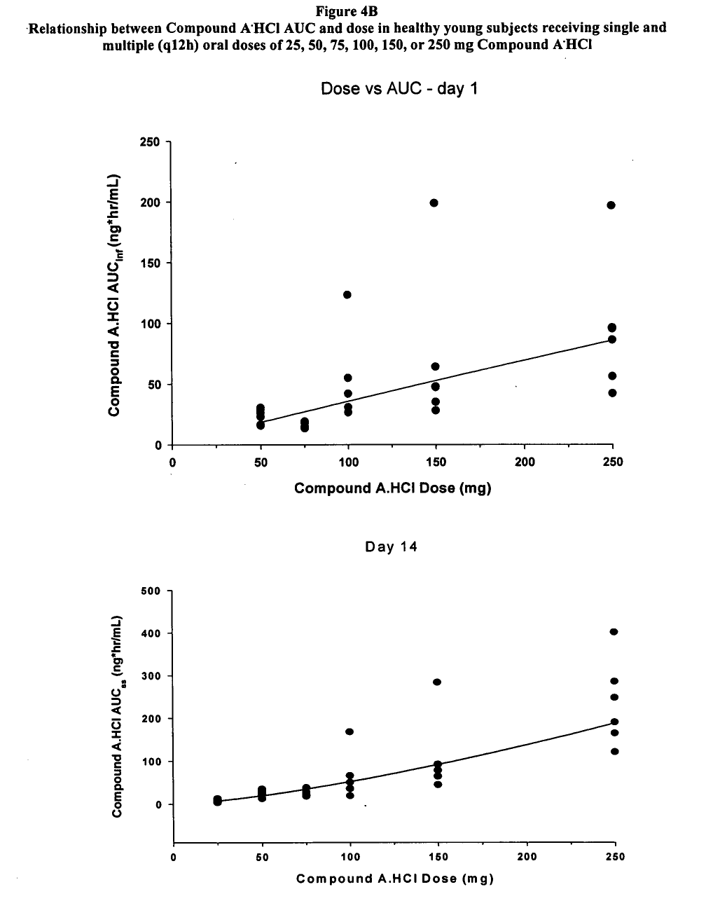

A total of 48 subjects contributed pharmacokinetic data for this analysis. Pharmacokinetic parameters for Compound A.HCl after single and multiple doses are shown in Tables 4A and 4B, respectively. FIG. 2 shows Compound A.HCl mean plasma concentration versus time profiles on days 1 and 14. FIG. 3 shows Compound A.HCl trough levels on days 7, 10 and 12 at various dose levels. FIGS. 4A and 4B show the relationship between Cₘₐₓ vs. dose and AUC vs. dose, respectively.

Following both single and multiple dose administration, Compound A.HCl was absorbed from the enteric-coated formulation with mean Tₘₐₓ ranging from 4 to 6 hours. The mean elimination t_{1/2} of Compound A.HCl in patients was 8 to 11 hours (single dose) and 6 to 8 hrs (multiple dose).

A minor secondary peak was seen at ˜24-28 hrs on day 14 in most of the subjects at all dose groups.

The relationship between Compound A.HCl Cₘₐₓ vs. dose and AUC vs. dose was described using the equation: Cₘₐₓ or AUC=a*(dose)^{b}. Compound A.HCl exhibited approximately linear dose-proportionality over the dose range of 25 to 150 mg q12h for single dose and steady-state AUC and Cₘₐₓ. However, slight greater-than-proportional increases in steady-state Cₘₐₓ and AUC were seen at the 250 mg q12h dose (approximately 2^{1.4} fold increase in AUC for a 2-fold increase in dose, approximately 2^{1.3} fold increase in Cₘₐₓ for a 2-fold increase in dose) with steady-state Cl/F being 54-60% lower at the 250 mg q12h dose in comparison to the lower dose groups. Graphical assessments of dose-proportionality are shown in FIGS. 4A and 4B.

TABLE 4A
Compound A.HCl pharmacokinetic parameters in healthy subjects
receiving a single oral dose of 25, 50, 75, 100,
150 or 250 mg Compound A.HCl

| | Dose | | Cₘₐₓ | Tₘₐₓ | t_{1/2} | AUC_{0-∞} |
|---|---|---|---|---|---|---|
| Day | (mg) | | (ng/mL) | (hr) | (hr) | (ng * hr/mL) |
| 1.00 | 25 | Mean | 0.87 | 5.2 | — | — |
| | | SD | 0.32 | 1.3 | — | — |
| | | CV % | 36.6 | 25.7 | — | — |
| | | Geom. | 0.81 | 5.0 | — | — |
| | | Mean | | | | |
| | 50 | Mean | 2.35 | 5.3 | 10.73 | 23.52 |
| | | SD | 0.56 | 1.6 | 5.89 | 6.16 |
| | | CV % | 23.9 | 30.6 | 54.9 | 26.2 |
| | | Geom. | 2.29 | 5.0 | 9.69 | 22.80 |
| | | Mean | | | | |
| | 75 | Mean | 1.60 | 5.5 | 8.22 | 16.94 |
| | | SD | 0.38 | 1.2 | 2.32 | 2.20 |
| | | CV % | 24.0 | 22.3 | 28.2 | 13.0 |
| | | Geom. | 1.56 | 5.3 | 7.93 | 16.81 |
| | | Mean | | | | |
| | 100 | Mean | 4.38 | 5.5 | 9.90 | 55.54 |
| | | SD | 2.85 | 1.2 | 3.76 | 39.43 |
| | | CV % | 65.0 | 22.3 | 37.9 | 71.0 |
| | | Geom. | 3.64 | 5.3 | 9.31 | 47.18 |
| | | Mean | | | | |
| | 150 | Mean | 5.05 | 5.7 | 9.08 | 70.23 |
| | | SD | 4.73 | 0.8 | 1.34 | 64.23 |
| | | CV % | 93.5 | 14.4 | 14.8 | 91.5 |
| | | Geom. | 3.67 | 5.6 | 9.00 | 55.27 |
| | | Mean | | | | |
| | 250 | Mean | 6.27 | 6.0 | 10.45 | 95.49 |
| | | SD | 2.73 | 1.3 | 3.43 | 54.27 |
| | | CV % | 43.5 | 21.1 | 32.8 | 56.8 |
| | | Geom. | 5.91 | 5.9 | 9.91 | 84.69 |
| | | Mean | | | | |

TABLE 4B
Compound A.HCl pharmacokinetic parameters in healthy subjects
receiving multiple oral doses (q12 h) of 25, 50, 75, 100, 150 or
250 mg Compound A.HCl

| | Dose | | Cₘₐₓ | Tₘₐₓ | t_{1/2} | *AUCss |
|---|---|---|---|---|---|---|
| Day | (mg) | | (ng/mL) | (hr) | (hr) | (ng * hr/mL) |
| 14.00 | 25 | Mean | 1.22 | 4.0 | 6.35 | 8.73 |
| | | SD | 0.37 | 1.9 | 1.20 | 3.71 |
| | | CV % | 30.5 | 46.8 | 18.9 | 42.6 |
| | | Geom. | 1.16 | 3.7 | 6.25 | 8.00 |
| | | Mean | | | | |
| | 50 | Mean | 3.39 | 5.7 | 7.64 | 26.49 |
| | | SD | 0.86 | 0.8 | 1.10 | 7.70 |
| | | CV % | 25.4 | 14.4 | 14.4 | 29.1 |
| | | Geom. | 3.27 | 5.6 | 7.58 | 25.32 |
| | | Mean | | | | |
| | 75 | Mean | 3.05 | 6.3 | 6.45 | 24.95 |
| | | SD | 0.94 | 0.8 | 1.05 | 7.50 |
| | | CV % | 31.0 | 12.9 | 16.4 | 30.0 |
| | | Geom. | 2.92 | 6.3 | 6.38 | 24.15 |
| | | Mean | | | | |
| | 100 | Mean | 7.81 | 5.5 | 7.86 | 62.76 |
| | | SD | 5.48 | 1.2 | 1.77 | 53.99 |
| | | CV % | 70.2 | 22.3 | 22.5 | 86.0 |
| | | Geom. | 6.45 | 5.3 | 7.72 | 49.18 |
| | | Mean | | | | |
| | 150 | Mean | 13.06 | 5.2 | 7.04 | 109.50 |
| | | SD | 10.93 | 1.3 | 0.80 | 87.16 |
| | | CV % | 83.7 | 25.7 | 11.4 | 79.6 |
| | | Geom. | 10.69 | 5.0 | 7.00 | 90.70 |
| | | Mean | | | | |
| | 250 | Mean | 26.82 | 6.2 | 7.12 | 234.75 |
| | | SD | 8.79 | 1.8 | 1.46 | 100.12 |
| | | CV % | 32.8 | 29.8 | 20.6 | 42.7 |
| | | Geom. | 25.64 | 5.9 | 6.99 | 217.81 |
| | | Mean | | | | |

*AUCₛₛ is AUC₀₋₁₂

### Example 7

### Administration of Enteric Coated Capsules Containing Compound A.HCl to Schizophrenic Patients

Ascending multiple doses of 100, 150, and 250 mg Compound A.HCl were administered as twice-daily regimen (q12h) for 10 days in cohorts of 8 schizophrenic patients (6 active and 2 placebo). Blood samples for Compound A.HCl analysis were obtained on day 1 within 2 hours of test article administration and at various time points from 0.5 to 24 hours after test article administration. On day 10, blood samples were collected up to 12 hours after dose administration. Doses were administered orally after an overnight fast of at least 10 hours on these 2 days. Trough PK samples were collected on days 6, 8, and 9 to assess whether steady-state has been reached.

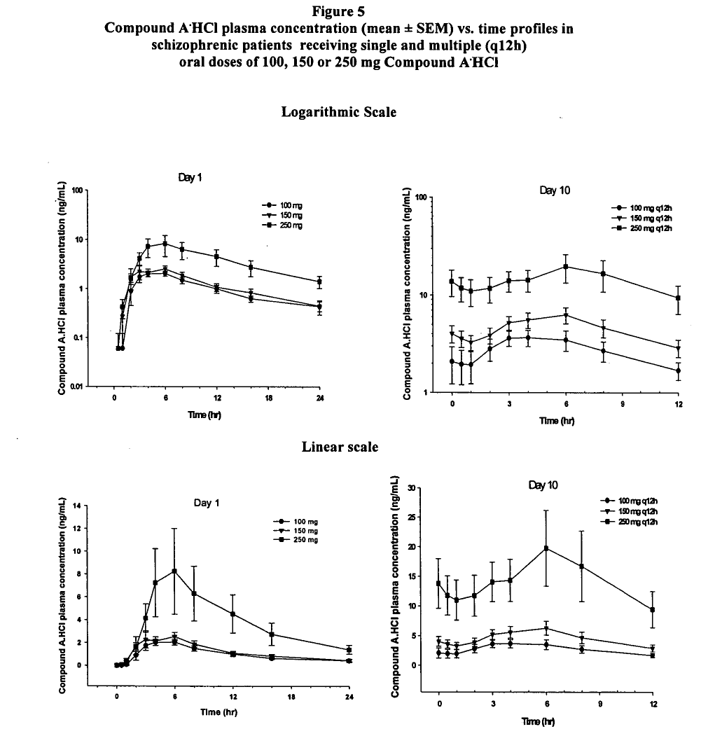

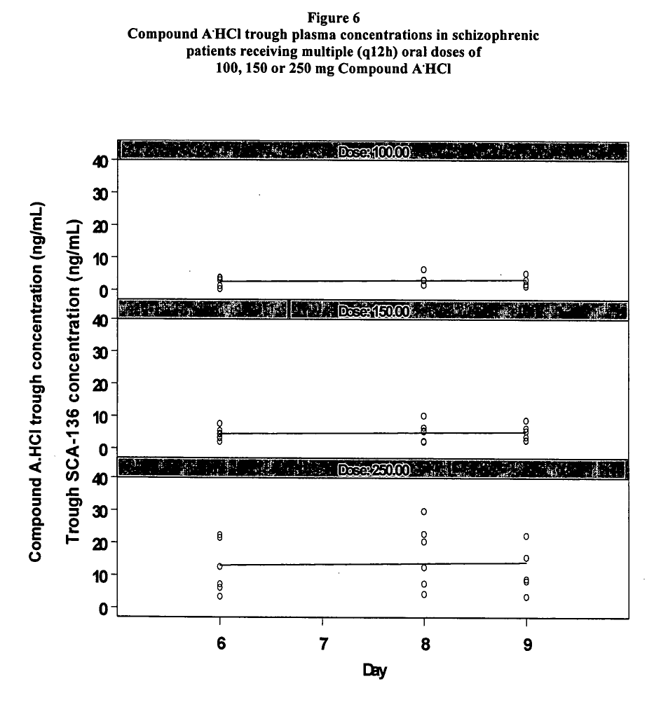

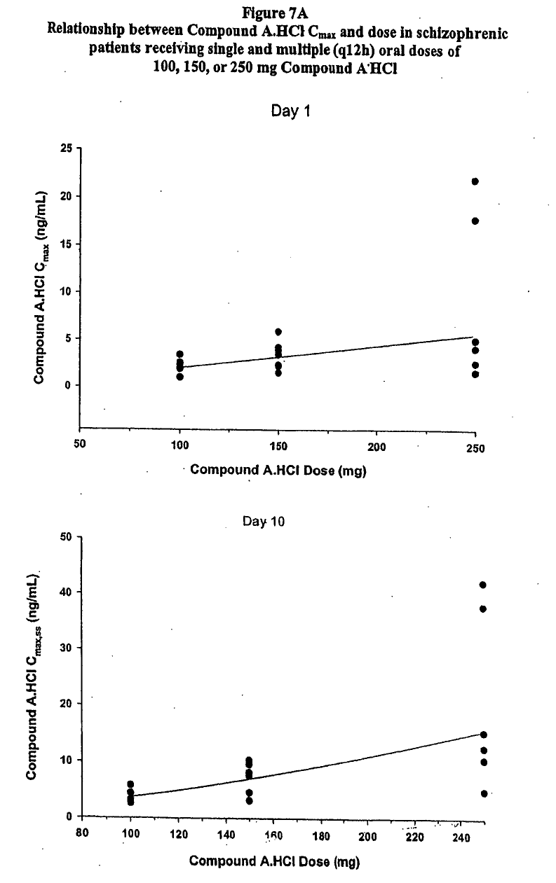

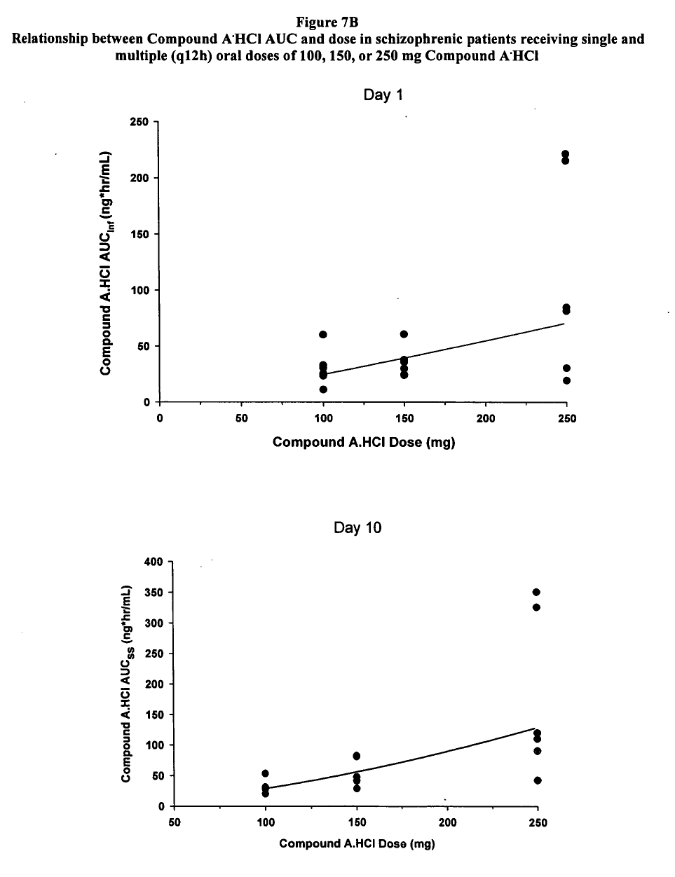

Pharmacokinetic parameters for Compound A.HCl after single and multiple doses are presented in Tables 5A and 5B, respectively. FIG. 5 shows Compound A.HCl mean plasma concentration versus time profiles on days 1 and 10. FIG. 6 shows Compound A.HCl trough levels on days 6, 8 and 9 at various dose levels. FIGS. 7A and 7B show the relationship between Cₘₐₓ vs. dose and AUC vs. dose, respectively.

Following both single and multiple dose administration to schizophrenic patients, Compound A.HCl was absorbed from the enteric-coated formulation with mean Tₘₐₓ ranging from 4 to 5 hours. The mean elimination t_{1/2} of Compound A.HCl in patients was 8 to 10 hours after single dose administration. Elimination t_{1/2} after multiple dose administration could not be accurately estimated as samples were colleted for up to 24 hours only.

Relationship between Compound A.HCl Cₘₐₓ vs. dose and AUC vs. dose was described using the equation: Cₘₐₓ or AUC=a*(dose)_{b}. Steady-state Cₘₐₓ and AUC appeared to increase in a greater-than-proportional manner at the 250 mg q12h dose (approximately 21.6 fold increase in AUC and Cₘₐₓ for a 2-fold increase in dose). The deviation from 1 in the exponent value was caused mostly by 2 patients (outliers) who had very high plasma concentrations in this dose group. The relationship between dose and exposure was approximately linear after excluding these 2 outlier patients from the analysis. Graphical assessments of dose-proportionality are shown in FIGS. 7A and 7B. These dose proportionality results are consistent with the results from the healthy subjects where greater-than-proportional increases in exposure were seen after multiple oral doses of 250 mg. Similar signs of nonlinearity were also seen in the single ascending dose study after single oral doses of 500 mg Compound A.HCl. There was no significant difference in Tₘₐₓ or t_{1/2} of Compound A.HCl at the 250 mg q12h dose level.

No significant differences in Compound A.HCl pharmacokinetics were seen between healthy subjects and schizophrenic patients.

TABLE 5A
Compound A.HCl pharmacokinetic parameters in Schizophrenia
patients receiving a single oral dose of 100, 150 or 250 mg
Compound A.HCl

| | Dose | | Cₘₐₓ | Tₘₐₓ | t_{1/2} | AUC_{0-∞} |
|---|---|---|---|---|---|---|
| Day | (mg) | | (ng/mL) | (hr) | (hr) | (ng * hr/mL) |
| 1.00 | 100 | N | 6 | 6 | 6 | 6 |
| | | Mean | 2.21 | 4.8 | 8.93 | 30.58 |
| | | SD | 0.81 | 1.3 | 2.69 | 16.37 |
| | | CV % | 36.6 | 27.5 | 30.2 | 53.5 |
| | | Geom. | 2.07 | 4.7 | 8.67 | 27.13 |
| | | Mean | | | | |
| | 150 | N | 7 | 7 | 7 | 7 |
| | | Mean | 3.37 | 4.6 | 9.49 | 35.60 |
| | | SD | 1.49 | 1.8 | 2.30 | 12.36 |
| | | CV % | 44.1 | 39.7 | 24.2 | 34.7 |
| | | Geom. | 3.09 | 4.2 | 9.25 | 34.04 |
| | | Mean | | | | |
| | 250 | N | 6 | 6 | 6 | 6 |
| | | Mean | 8.89 | 5.0 | 8.04 | 108.80 |
| | | SD | 8.75 | 2.0 | 2.56 | 88.91 |
| | | CV % | 98.4 | 40.0 | 31.8 | 81.7 |
| | | Geom. | 5.71 | 4.7 | 7.74 | 76.09 |
| | | Mean | | | | |

TABLE 5B
Compound A.HCl pharmacokinetic parameters in Schizophrenia
patients receiving multiple oral doses (q12 h) of 100, 150 or
250 mg Compound A.HCl

| | | | Cₘₐₓ | Tₘₐₓ | *AUCss |
|---|---|---|---|---|---|
| Day | Dose (mg) | | (ng/mL) | (hr) | (ng * hr/mL) |
| 10.00 | 100 | N | 4 | 4 | 4 |
| | | Mean | 4.00 | 4.8 | 33.23 |
| | | SD | 1.39 | 1.5 | 14.20 |
| | | CV % | 34.7 | 31.6 | 42.7 |
| | | Geom. Mean | 3.82 | 4.6 | 31.19 |
| | 150 | N | 6 | 6 | 6 |
| | | Mean | 7.21 | 4.3 | 54.88 |
| | | SD | 2.79 | 1.4 | 22.01 |
| | | CV % | 38.7 | 31.5 | 40.1 |
| | | Geom. Mean | 6.67 | 4.2 | 51.27 |
| | 250 | N | 6 | 6 | 6 |
| | | Mean | 20.60 | 4.8 | 173.49 |
| | | SD | 15.51 | 2.9 | 130.74 |
| | | CV % | 75.3 | 59.1 | 75.4 |
| | | Geom. Mean | 15.91 | NC | 134.35 |

*AUCₛₛ is AUC₀₋₁₂
NC = not calculated as 1 subject had peak concentrations at time = 0.

It is intended that each of the patents, applications, and printed publications including books mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A pharmaceutical composition comprising:
a) a pharmaceutically effective amount of an active pharmacological agent comprising from about 10% to about 80% by weight of the pharmaceutical composition;
b) a filler component comprising from about 10% to about 80% by weight of the pharmaceutical composition;
c) an optional seal coat component comprising from about 0.01% to about 5% by weight of the pharmaceutical composition;
d) an enteric coat component comprising from about 0.01% to about 20% by weight of the pharmaceutical composition;
e) an optional glidant component comprising from about 0.01% to about 20% by weight of the pharmaceutical composition;
f) an optional plasticizer component comprising from about 0.01% to about 3% by weight of the pharmaceutical composition;
g) an optional neutralizer component comprising from about 0.01% to about 1.5% by weight of the pharmaceutical composition;
h) an optional surfactant component comprising from about 0.001% to about 1.0% by weight of the pharmaceutical composition; and
i) an optional lubricant component comprising from about 0.01% to about 5.0% by weight of the pharmaceutical composition;
wherein the active pharmacological agent has the Formula I: 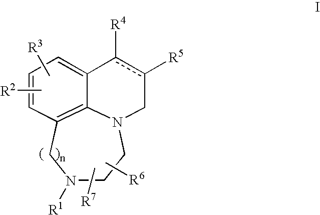
wherein:
R¹ is hydrogen, alkyl of 1 to 6 carbon atoms, alkanoyl of 2 to 6 carbon atoms, or carboarylalkoxy of 7 to 11 carbon atoms;
R² and R³ are each, independently, hydrogen, hydroxy, alkyl of 1-6 carbon atoms, alkoxy of 1-6 carbon atoms, halogen, carboxamido, carboalkoxy of two to six carbon atoms, perfluoroalkyl of 1-6 carbon atoms, cyano, alkanesulfonamido of 1-6 carbon atoms, alkanesulfonyl of 1-6 carbon atoms, alkanamido of 1-6 carbon atoms, amino, alkylamino of 1-6 carbon atoms, dialkylamino of 1-6 carbon atoms per alkyl moiety, perfluoroalkoxy of 1-6 carbon atoms, alkanoyloxy of 2 to 6 carbon atoms, alkanoyl of 2 to 6 carbon atoms, aroyl of 6 to 8 carbon atoms, aryl of 5 to 7 carbon atoms, a C₆ to C₁₃ alkylaryl group having 5 to 7 carbon atoms in the aryl moiety, a 5 to 7 membered heteroaryl group, or a 6 to 13 membered alkylheteroaryl group having 5 to 7 members in the heteroaryl moiety, wherein any R² or R³ substituent having an aryl or heteroaryl moiety may optionally be substituted on the aryl or heteroaryl moiety with 1 to 3 substituents independently selected from a halogen atom, a C₁-C₆ alkyl group, or a C₁-C₆ alkoxy group;
R⁴ and R⁵ are, independently, hydrogen or alkyl of 1 to 6 carbon atoms, or R⁴ and R⁵, taken together with the carbons to which they are attached, form a cyclic moiety selected from a cycloalkane of 4 to 8 carbon atoms, cycloalkene of 4 to 8 carbon atoms, bridged bicyclic alkane of 5 to 10 carbon atoms, bridged bicyclic alkene of 5 to 10 carbon atoms, pyran or thiopyran in which the sulfur atom is optionally oxidized to the sulfoxide or sulfone, wherein the cyclic moiety formed by R⁴ and R⁵ may optionally be substituted with 1 to 3 substituents independently selected from a halogen atom, a C₁-C₆ alkyl group, or a C₁-C₆ alkoxy group;
R⁵ and R⁷ are each, independently, hydrogen or alkyl of 1 to 6 carbon atoms;
n is 1 or 2; and
a dotted line represents an optional double bond;
or a pharmaceutically acceptable salt thereof.

2. The pharmaceutical composition of claim 1, wherein R² and R³ are independently selected from hydrogen, halo, trifluoromethyl, phenyl or alkoxy of 1 to 3 carbon atoms, R¹, R⁵ and R⁷ are each hydrogen, n is 1, and R⁴ and R⁵, taken together with the carbon atoms to which they are attached, form cyclopentane, cyclohexane or cycloheptane.

3. The pharmaceutical composition of claim 1, wherein said active pharmacological agent is (9aR,12aS)-4,5,6,7,9,9a,10,11,12,12a-decahydrocyclopenta[c][1,4]diazepino[6,7,1-ij]quinoline hydrochloride.

4. The pharmaceutical composition of claim 3, wherein:
a) the active pharmacological agent comprises from about 15% to about 25% by weight of the pharmaceutical composition;
b) the filler component comprises from about 50% to about 70% by weight of the pharmaceutical composition;
c) the optional seal coat component, when present, comprises from about 0.5% to about 3% by weight of the pharmaceutical composition;
d) the enteric coat component comprises from about 5% to about 15% by weight of the pharmaceutical composition;
e) the optional glidant component, when present, comprises from about 0.01% to about 1% by weight of the pharmaceutical composition;
f) the optional plasticizer component, when present, comprises from about 0.1% to about 1.5% by weight of the pharmaceutical composition;
g) the optional neutralizer component, when present, comprises from about 0.01% to about 0.8% by weight of the pharmaceutical composition;
h) the optional surfactant component, when present, comprises from about 0.005% to about 0.05% by weight of the pharmaceutical composition; and
i) the optional lubricant component, when present, comprises from about 1% to about 4% by weight of the pharmaceutical composition.

5. The pharmaceutical composition of claim 3, wherein:
a) the active pharmacological agent comprises from about 30% to about 40% by weight of the pharmaceutical composition;
b) the filler component comprises from about 40% to about 50% by weight of the pharmaceutical composition;
c) the optional seal coat component, when present, comprises from about 2% to about 3% by weight of the pharmaceutical composition;
d) the enteric coat component comprises from about 12% to about 16% by weight of the pharmaceutical composition;
e) the optional glidant component, when present, comprises from about 0.1% to about 3% by weight of the pharmaceutical composition;
f) the optional plasticizer component, when present, comprises from about 0.5% to about 2.0% by weight of the pharmaceutical composition;
g) the optional neutralizer component, when present, comprises from about 0.03% to about 0.3% by weight of the pharmaceutical composition;
h) the optional surfactant component, when present, comprises from about 0.001% to about 0.3% by weight of the pharmaceutical composition; and
i) the optional lubricant component, when present, comprises from about 2.0% to about 3.5% by weight of the pharmaceutical composition.

6. The pharmaceutical composition of claim 3, wherein the glidant component, the plasticizer component, the neutralizer component, the surfactant component, and the lubricant component are each present.

7. The pharmaceutical composition of claim 3, wherein:
the filler component comprises one or more of microcrystalline cellulose, lactose, starch, carboxymethyl cellulose, cellulose gum, polyethylene glycol, substituted celluloses, ethyl cellulose, carboxyethyl cellulose, hydroxyethyl celluloses, calcium phosphates, anhydrous dicalcium phosphate, metal aluminosilicates, magnesium aluminometasilicate, sugar or carbohydrate containing compounds, mannitol, sucrose, maltodextrin, sorbitol, starch, xylitol, metal phosphates, metal carbonates, and magnesium carbonate;
the enteric coat component comprises one or more of a methacrylic copolymer, methacrylic acid co-polymer, a HPMC containing enteric coating system, CAP, HPMCP, an acrylic polymer, or other acrylate-methacrylate or cellulose acetate phthalate based coat;
the optional glidant component, when present, comprises one or more of mono- and di-glycerides, talc, silicon dioxide, stearic acid, starch, powdered cellulose, lactose, stearates, calcium phosphates, magnesium carbonate, magnesium oxide, silicates, and silicon dioxide aerogels;
the optional plasticizer component, when present, comprises one or more of triethyl citrate, dibutyl sebecate, polyethylene glycol, propylene glycol, triacetin, sorbitol, tributyl citrate, acetyltributyl citrate, acetyltriethyl citrate, dibutyl phthalate, triethyl citrate and triethanolamine;
the optional neutralizer component, when present, comprises one or more of NaOH, KOH and NH₄OH;
the optional surfactant component, when present, comprises one or more of polysorbate 80, sodium lauryl sulfate, sucrose palmitate, poloxamer, docusate sodium, and polyoxyethylene sorbitan fatty acid esters, polyoxyethylene stearates, sucrose fatty acid esters and sorbitan fatty acid esters; and
the optional lubricant component, when present, comprises one or more of talc, metallic stearates, silicon dioxide, sodium stearyl fumarate, fatty acid esters, fatty acids, fatty alcohols, glyceryl behenate, mineral oil, paraffins, hydrogenated vegetable oils, leucine, polyethylene glycols, metallic lauryl sulfates, Aerosil 200, and sodium chloride.

8. The pharmaceutical composition of claim 7, wherein the glidant component, the plasticizer component, the neutralizer component, the surfactant component, and the lubricant component are each present.

9. The pharmaceutical composition of claim 3, wherein:
the filler component comprises microcrystalline cellulose;
the enteric coat component comprises a methyacrylic copolymer with an anionic functional group;
the optional glidant component, when present, comprises mono- and di-glycerides;
the optional plasticizer component, when present, comprises triethyl citrate;
the optional neutralizer component, when present, comprises NaOH;
the optional surfactant component, when present, comprises polysorbate 80; and
the lubricant component, when present, comprises talc.

10. The pharmaceutical composition of claim 3, wherein the composition contains from about 0.5 mg to about 5.0 mg of active pharmacological agent.

11. The pharmaceutical composition of claim 3, wherein the composition contains from about 20 mg to about 110 mg of active pharmacological agent.

12. The pharmaceutical composition of claim 3, wherein the composition comprises a plurality of enteric-coated pellets.

13. The pharmaceutical composition of claim 12, wherein said enteric-coated pellets are present in a capsule.

14. The pharmaceutical composition of claim 7, wherein the composition comprises a plurality of enteric-coated pellets.

15. The pharmaceutical composition of claim 14, wherein said enteric-coated pellets are present in a capsule.

16. A process for preparing a pharmaceutical composition comprising a plurality of enteric-coated pellets, said pellets comprising a composition of claim 11, the process comprising:
a) preparing uncoated pellets comprising said filler and said active pharmacological agent;
b) optionally applying a subcoat to the uncoated pellets; and
c) applying an enteric coating to the pellets.

17. The process of claim 16, further comprising the step of:
d) filling a capsule with said pellets to achieve a predetermined dose of (9aR,12aS)-4,5,6,7,9,9a,10,11,12,12a-decahydrocyclopenta[c][1,4]diazepino[6,7,1-ij]quinoline hydrochloride.

18. The process of claim 16, wherein said step (a) comprises:
i) mixing said filler and said active pharmacological agent to form a mixture;
ii) wet granulating the mixture to form a granulate; and
iii) extruding and spheronizing the granulate.

19. The process of claim 16, wherein said step (c) comprises:
i) preparing a suspension comprising said enteric coat, said plasticizer, said neutralizer, and said surfactant; and
ii) spraying said suspension onto said pellets.

20. The product of the process of claim 16.

21. The process of claim 16, wherein the composition contains from about 0.5 mg to about 5.0 mg of active pharmacological agent.

22. The process of claim 16, wherein the composition contains from about 20 mg to about 110 mg of active pharmacological agent.

23. A pharmaceutical composition comprising a plurality of enteric coated pellets, said pellets comprising:
a) an active pharmacological agent comprising from about 20% to about 40% by weight of the pharmaceutical composition;
b) a filler component comprising from about 40% to about 70% by weight of the pharmaceutical composition;
c) an optional seal coat component comprising, when present, from about 1% to about 3% by weight of the pharmaceutical composition;
d) an enteric coat component comprising from about 8% to about 16% by weight of the pharmaceutical composition;
e) an optional glidant component comprising, when present, from about 0.1% to about 3% by weight of the pharmaceutical composition;
f) an optional plasticizer component comprising, when present, from about 0.5% to about 2.0% by weight of the pharmaceutical composition;
g) an optional neutralizer component comprising, when present, from about 0.03% to about 0.3% by weight of the pharmaceutical composition;
h) an optional surfactant component comprising, when present, from about 0.001% to about 0.3% by weight of the pharmaceutical composition; and
i) an optional lubricant component comprising, when present, from about 2.0% to about 3.5% by weight of the pharmaceutical composition;
wherein the active pharmacological agent comprises (9aR,12aS)-4,5,6,7,9,9a,10,11,12,12a-decahydrocyclopenta[c][1,4]diazepino[6,7,1-ij]quinoline hydrochloride.

24. The pharmaceutical composition of claim 3, wherein:
a) the active pharmacological agent comprises from about 30% to about 45% by weight of the pharmaceutical composition;
b) the filler component comprises from about 40% to about 60% by weight of the pharmaceutical composition;
c) the optional seal coat component, when present, comprises from about 0.5% to about 3% by weight of the pharmaceutical composition;
d) the enteric coat component comprises from about 5% to about 15% by weight of the pharmaceutical composition;
e) the optional glidant component, when present, comprises from about 0.1% to about 2% by weight of the pharmaceutical composition;
f) the optional plasticizer component, when present, comprises from about 0.1% to about 1.5% by weight of the pharmaceutical composition;
g) the optional neutralizer component, when present, comprises from about 0.01% to about 0.8% by weight of the pharmaceutical composition;
h) the optional surfactant component, when present, comprises from about 0.005% to about 0.05% by weight of the pharmaceutical composition; and
i) the optional lubricant component, when present, comprises from about 1% to about 4% by weight of the pharmaceutical composition.

25. The pharmaceutical composition of claim 3, wherein:
a) the active pharmacological agent comprises from about 50% to about 70% by weight of the pharmaceutical composition;
b) the filler component comprises from about 10% to about 30% by weight of the pharmaceutical composition;
c) the optional seal coat component, when present, comprises from about 0.5% to about 3% by weight of the pharmaceutical composition;
d) the enteric coat component comprises from about 5% to about 15% by weight of the pharmaceutical composition;
e) the optional glidant component, when present, comprises from about 0.1% to about 2% by weight of the pharmaceutical composition;
f) the optional plasticizer component, when present, comprises from about 0.1% to about 1.5% by weight of the pharmaceutical composition;
g) the optional neutralizer component, when present, comprises from about 0.01% to about 0.8% by weight of the pharmaceutical composition;
h) the optional surfactant component, when present, comprises from about 0.005% to about 0.05% by weight of the pharmaceutical composition; and
i) the optional lubricant component, when present, comprises from about 1% to about 4% by weight of the pharmaceutical composition.

26. The pharmaceutical composition of claim 3, wherein:
a) the active pharmacological agent comprises from about 60% to about 70% by weight of the pharmaceutical composition;
b) the filler component comprises from about 10% to about 30% by weight of the pharmaceutical composition;
c) the optional seal coat component, when present, comprises from about 0.5% to about 3% by weight of the pharmaceutical composition;
d) the enteric coat component comprises from about 5% to about 15% by weight of the pharmaceutical composition;
e) the optional glidant component, when present, comprises from about 0.1% to about 2% by weight of the pharmaceutical composition;
f) the optional plasticizer component, when present, comprises from about 0.1% to about 1.5% by weight of the pharmaceutical composition;
g) the optional neutralizer component, when present, comprises from about 0.01% to about 0.8% by weight of the pharmaceutical composition;
h) the optional surfactant component, when present, comprises from about 0.005% to about 0.05% by weight of the pharmaceutical composition; and
i) the optional lubricant component, when present, comprises from about 1% to about 4% by weight of the pharmaceutical composition.

27. The pharmaceutical composition of claim 25, wherein the composition contains from about 50 mg to about 750 mg of active pharmacological agent.

28. A pharmaceutical composition comprising (9aR,12aS)-4,5,6,7,9,9a,10,11,12,12a-decahydrocyclopenta[c][1,4]diazepino[6,7,1-ij]quinoline hydrochloride in an enteric coated form.

29. The pharmaceutical composition of claim 28, further comprising a sustained release coating.

30. A pharmaceutical composition comprising (9aR,12aS)-4,5,6,7,9,9a,10,11,12,12a-decahydrocyclopenta[c][1,4]diazepino[6,7,1-ij]quinoline hydrochloride, said composition being effective to provide a mean plasma concentration profile for (9aR,12aS)-4,5,6,7,9,9a,10,11,12,12a-decahydrocyclopenta[c][1,4]diazepino[6,7,1-ij]quinoline in human schizophrenia patients which has a mean AUCss of about 33.23 hr*ng/mL±20% for a dosage of 100 mg (9aR,12aS)-4,5,6,7,9,9a,10,11,12,12a-decahydrocyclopenta[c][1,4]diazepino[6,7,1-ij]quinoline hydrochloride, or a respective mean AUC value about proportional thereto for a total dose other than 100 mg.

31. The pharmaceutical composition of claim 30 wherein said (9aR, 12aS)-4,5,6,7,9,9a,10,11,12,12a-decahydrocyclopenta[c][1,4]diazepino[6,7,1-ij]quinoline hydrochloride is present in an enteric coated form.

32. A pharmaceutical composition comprising (9aR,12aS)-4,5,6,7,9,9a,10,11,12,12a-decahydrocyclopenta[c][1,4]diazepino[6,7,1-ij]quinoline hydrochloride, said composition being effective to provide a mean plasma concentration profile for (9aR,12aS)-4,5,6,7,9,9a,10,11,12,12a-decahydrocyclopenta[c][1,4]diazepino[6,7,1-ij]quinoline in human schizophrenia patients which has a mean AUCss of about 54.88 hr*ng/mL±20% for a dosage of 150 mg (9aR,12aS)-4,5,6,7,9,9a,10,11,12,12a-decahydrocyclopenta[c][1,4]diazepino[6,7,1-ij]quinoline hydrochloride, or a respective mean AUC value about proportional thereto for a total dose other than 150 mg.

33. The pharmaceutical composition of claim 32 wherein said (9aR, 12aS)-4,5,6,7,9,9a,10,11,12,12a-decahydrocyclopenta[c][1,4]diazepino[6,7,1-ij]quinoline hydrochloride is present in an enteric coated form.

34. A pharmaceutical composition comprising (9aR,12aS)-4,5,6,7,9,9a,10,11,12,12a-decahydrocyclopenta[c][1,4]diazepino[6,7,1-ij]quinoline hydrochloride, said composition being effective to provide a mean plasma concentration profile for (9aR,12aS)-4,5,6,7,9,9a,10,11,12,12a-decahydrocyclopenta[c][1,4]diazepino[6,7,1-ij]quinoline in human schizophrenia patients which has a mean AUCss of about 173.49 hr*ng/mL±20% for a dosage of 250 mg (9aR,12aS)-4,5,6,7,9,9a,10,11,12,12a-decahydrocyclopenta[c][1,4]diazepino[6,7,1ij]-quinoline hydrochloride, or a respective mean AUC value about proportional thereto for a total dose other than 250 mg.

35. The pharmaceutical composition of claim 34 wherein said (9aR, 12aS)-4,5,6,7,9,9a,10,11,12,12a-decahydrocyclopenta[c][1,4]diazepino[6,7,1-ij]quinoline hydrochloride is present in an enteric coated form.

36. A pharmaceutical composition comprising (9aR,12aS)-4,5,6,7,9,9a,10,11,12,12a-decahydrocyclopenta[c][1,4]diazepino[6,7,1-ij]quinoline hydrochloride, said composition being effective to provide a mean plasma concentration profile for (9aR,12aS)-4,5,6,7,9,9a,10,11,12,12a-decahydrocyclopenta[c][1,4]diazepino[6,7,1-ij]quinoline in healthy humans which has a mean AUCss of about 62.76 hr*ng/mL±20% for a dosage of 100 mg (9aR,12aS)-4,5,6,7,9,9a,10,11,12,12a-decahydrocyclopenta[c][1,4]diazepino[6,7,1-ij]quinoline hydrochloride, or a respective mean AUC value about proportional thereto for a total dose other than 100 mg.

37. The pharmaceutical composition of claim 36 wherein said (9aR, 12aS)-4,5,6,7,9,9a,10,11,12,12a-decahydrocyclopenta[c][1,4]diazepino[6,7,1-ij]quinoline hydrochloride is present in an enteric coated form.

38. A pharmaceutical composition comprising (9aR,12aS)-4,5,6,7,9,9a,10,11,12,12a-decahydrocyclopenta[c][1,4]diazepino[6,7,1-ij]quinoline hydrochloride, said composition being effective to provide a mean plasma concentration profile for (9aR,12aS)-4,5,6,7,9,9a,10,11,12,12a-decahydrocyclopenta[c][1,4]diazepino[6,7,1-ij]quinoline in healthy humans which has a mean AUCss of about 109.50 hr*ng/mL±20% for a dosage of 150 mg (9aR, 12aS)-4,5,6,7,9,9a,10,11,12,12a-decahydrocyclopenta[c][1,4]diazepino[6,7,1-ij]quinoline hydrochloride, or a respective mean AUC value about proportional thereto for a total dose other than 150 mg.

39. The pharmaceutical composition of claim 38 wherein said (9aR, 12aS)-4,5,6,7,9,9a,10,11,12,12a-decahydrocyclopenta[c][1,4]diazepino[6,7,1-ij]quinoline hydrochloride is present in an enteric coated form.

40. A pharmaceutical composition comprising (9aR,12aS)-4,5,6,7,9,9a,10,11,12,12a-decahydrocyclopenta[c][1,4]diazepino[6,7,1-ij]quinoline hydrochloride, said composition being effective to provide a mean plasma concentration profile for (9aR,12aS)-4,5,6,7,9,9a,10,11,12,12a-decahydrocyclopenta[c][1,4]diazepino[6,7,1-ij]quinoline in healthy humans which has a mean AUCss of about 234.75 hr*ng/mL±20% for a dosage of 250 mg (9aR,12aS)-4,5,6,7,9,9a,10,11,12,12a-decahydrocyclopenta[c][1,4]diazepino[6,7,1-ij]quinoline hydrochloride, or a respective mean AUC value about proportional thereto for a total dose other than 250 mg.

41. The pharmaceutical composition of claim 40 wherein said (9aR, 12aS)-4,5,6,7,9,9a,10,11,12,12a-decahydrocyclopenta[c][1,4]diazepino[6,7,1-ij]quinoline hydrochloride is present in an enteric coated form.

42. A pharmaceutical composition comprising (9aR,12aS)-4,5,6,7,9,9a,10,11,12,12a-decahydrocyclopenta[c][1,4]diazepino[6,7,1-ij]quinoline hydrochloride, said composition being effective to provide a Tₘₐₓ of from about 4 hours to about 6.5 hours in healthy humans or in a patient.

43. A unit dosage form comprising from about 2 mg to about 150 mg of (9aR,12aS)-4,5,6,7,9,9a,10,11,12,12a-decahydrocyclopenta[c][1,4]diazepino[6,7,1-ij]quinoline hydrochloride, the dosage form providing a Cₘₐₓ of (9aR,12aS)-4,5,6,7,9,9a,10,11,12,12a-decahydrocyclopenta[c][1,4]diazepino[6,7,1-ij]quinoline between about 4 and about 8 hours after administration to a subject.

44. A unit dosage form comprising an amount of (9aR,12aS)-4,5,6,7,9,9a,10,11,12,12a-decahydrocyclopenta[c][1,4]diazepino[6,7,1-ij]quinoline hydrochloride, the unit dosage form characterized in that the dosage form comprises a degradable coating, characterized in that the coating degrades so that less than 30% of the (9aR,12aS)-4,5,6,7,9,9a,10,11,12,12a-decahydrocyclopenta[c][1,4]diazepino[6,7,1-ij]quinoline hydrochloride is released after two hours.

45. A unit dosage form of a medicament, comprising:
(9aR,12aS)-4,5,6,7,9,9a,10,11,12,12a-decahydrocyclopenta[c][1,4]diazepino[6,7,1-ij]quinoline hydrochloride; and
a degradable coating, characterized in that the coating degrades so that less than 30% of the (9aR,12aS)-4,5,6,7,9,9a,10,11,12,12a-decahydrocyclopenta[c][1,4]diazepino[6,7,1-ij]quinoline is released after two hours.

46. A unit dosage form of a medicament, comprising:
(9aR,12aS)-4,5,6,7,9,9a,10,11,12,12a-decahydrocyclopenta[c][1,4]diazepino[6,7,1-ij]quinoline hydrochloride; and
a degradable coating, characterized in that the coating degrades so that (9aR,12aS)-4,5,6,7,9,9a,10,11,12,12a-decahydrocyclopenta[c][1,4]diazepino[6,7,1-ij]quinoline hydrochloride is released to provide a Cₘₐₓ of (9aR,12aS)-4,5,6,7,9,9a,10,11,12,12a-decahydrocyclopenta[c][1,4]diazepino[6,7,1-ij]quinoline between about 4 abd about 8 hours after administration.

47. A unit dosage form of a medicament having a uniform dosage of (9aR, 12aS)-4,5,6,7,9,9a,10,11,12,12a-decahydrocyclopenta[c][1,4]diazepino[6,7,1-ij]quinoline hydrochloride, the dosage form being characterized by a dissolution profile upon oral administration in which the (9aR,12aS)-4,5,6,7,9,9a,10,11,12,12a-decahydrocyclopenta[c][1,4]diazepino[6,7,1-ij]quinoline hydrochloride is released such that a Cₘₐₓ of (9aR,12aS)-4,5,6,7,9,9a,10,11,12,12a-decahydrocyclopenta[c][1,4]diazepino[6,7,1-ij]quinoline occurs between about 4 and about 8 hours after administration to a subject.

48. The unit dosage form of claim 47, wherein said dosage form comprises a plurality of enterically coated pellets.

49. A method of preparing a formulation comprising (9aR,12aS)-4,5,6,7,9,9a,10,11,12,12a-decahydrocyclopenta[c][1,4]diazepino[6,7,1-ij]quinoline hydrochloride, the method comprising steps of:
providing pellets comprising (9aR,12aS)-4,5,6,7,9,9a,10,11,12,12a-decahydrocyclopenta[c][1,4]diazepino[6,7,1-ij]quinoline hydrochloride; and
applying to the pellets an enteric coating comprising an enteric coating polymer, in an amount that provides a weight gain of enteric coating polymer of from about 12% to about 22% relative to the weight of the uncoated pellets.

50. A method of preparing a formulation comprising (9aR,12aS)-4,5,6,7,9,9a,10,11,12,12a-decahydrocyclopenta[c][1,4]diazepino[6,7,1-ij]quinoline hydrochloride, the method comprising steps of:
providing pellets comprising (9aR,12aS)-4,5,6,7,9,9a,10,11,12,12a-decahydrocyclopenta[c][1,4]diazepino[6,7,1-ij]quinoline hydrochloride; and
applying to the pellets an enteric coating, wherein the coating degrades after administration of the formulation, such that the (9aR,12aS)-4,5,6,7,9,9a,10,11,12,12a-decahydrocyclopenta[c][1,4]diazepino[6,7,1-ij]quinoline hydrochloride is released such that a Cₘₐₓ of (9aR,12aS)-4,5,6,7,9,9a,10,11,12,12a-decahydrocyclopenta[c][1,4]diazepino[6,7,1-ij]quinoline occurs between about 4 and about 8 hours after administration to a subject.

51. The method of claim 50, wherein the coating achieves a dissolution profile characterized by less than 30% release of (9aR,12aS)-4,5,6,7,9,9a,10,11,12,12a-decahydrocyclopenta[c][1,4]diazepino[6,7,1-ij]quinoline hydrochloride after 2 hours.

52. A formulation comprising (9aR,12aS)-4,5,6,7,9,9a,10,11,12,12a-decahydrocyclopenta[c][1,4]diazepino[6,7,1-ij]quinoline hydrochloride, wherein the formulation provides a blood serum level of (9aR,12aS)-4,5,6,7,9,9a,10,11,12,12a-decahydrocyclopenta[c][1,4]diazepino[6,7,1-ij]quinoline that is characterized by a maximum peak, followed by a secondary peak of lesser value.

53. The formulation of claim 52, wherein the (9aR,12aS)-4,5,6,7,9,9a,10,11,12,12a-decahydrocyclopenta[c][1,4]diazepino[6,7,1-ij]quinoline hydrochloride is present in an enteric coated form.

54. A formulation comprising (9aR,12aS)-4,5,6,7,9,9a,10,11,12,12a-decahydrocyclopenta[c][1,4]diazepino[6,7,1-ij]quinoline hydrochloride, wherein the formulation provides release of (9aR,12aS)-4,5,6,7,9,9a,10,11,12,12a-decahydrocyclopenta[c][1,4]diazepino[6,7,1-ij]quinoline hydrochloride at a pH of greater than about 5.

55. A formulation comprising (9aR,12aS)-4,5,6,7,9,9a,10,11,12,12a-decahydrocyclopenta[c][1,4]diazepino[6,7,1-ij]quinoline hydrochloride, wherein the release of (9aR,12aS)-4,5,6,7,9,9a,10,11,12,12a-decahydrocyclopenta[c][1,4]diazepino[6,7,1-ij]quinoline hydrochloride after administration to a subject is delayed.
